(19) United States
(12) Patent Application Publication
(10) Pub. No.: US 2023/0002344 A1
(43) Pub. Date: Jan. 5, 2023

(54) NOVEL BENZOTHIOPHENE DERIVATIVES AND USE THEREOF FOR STIMULATING MITOCHONDRIAL TURNOVER

(71) Applicant: NIBN, The National Institute For Biotechnology In The Negev Ltd., Beer Sheva (IL)

(72) Inventors: Guy Las, Rehovot (IL); Arie Lev Gruzman, Jerusalem (IL); Femanda Cerqueira, Winthrop, MA (US); Orian S. Shirihai, Los Angeles, CA (US)

(21) Appl. No.: 17/777,496

(22) PCT Filed: Nov. 18, 2020
(86) PCT No.: PCT/IB2020/060872

§ 371 Date: May 17, 2022

Related U.S. Application Data
(60) Provisional application No. 62/937,532, filed on Nov. 19, 2019.

(51) Int. Cl. C07D 333/58; C07D 413/06; C07D 471/04; C07D 487/04; A61P 3/10

(52) U.S. Cl. CPC C07D 333/58; C07D 413/06; C07D 471/04; C07D 487/04; A61P 3/10

(57) ABSTRACT

Small organic compounds are described that stimulate mitochondrial clearance and are useful for the treatment of diseases associated with impaired mitochondrial turnover. Pharmaceutical compositions comprising these compounds and methods of using the compounds and their pharmaceutical compositions are provided, such as for treating Parkinson's disease, Type-2-diabetes, Huntington's disease, Alzheimer's disease and dementia.

### CROSS-REFERENCE TO RELATED APPLICATION

This application claims priority to U.S. Provisional Application Ser. No. 62/937,532, filed Nov. 19, 2019, which is incorporated herein by reference in its entirety.

### BACKGROUND

Normal physiology relies on maintaining mitochondrial mass and function by a continuous balance between mitochondrial biogenesis and disposal, termed ‘mitochondrial turnover’. Impaired mitochondrial turnover is increasingly acknowledged to be a central factor in aging and in the etiology of several age-associated diseases (including Parkinson, Huntington's, and Alzheimer) (Terman, A., et al., Mitochondrial turnover and aging of long-lived postmitotic cells: the mitochondrial-lysosomal axis theory of aging. Antioxid Redox Signal, 2010. 12 (4): p. 503-35.). Recent evidence indicate that in type-2-diabetes (T2D) as well, mitochondrial turnover is suppressed, leading to the accumulation of damaged mitochondria, which results in beta-cell dysfunction and apoptosis (Las, G., et al., Fatty acids suppress autophagic turnover in beta-cells. J Biol Chem, 2011. 286 (49): p. 42534-44; Masini, M., et al., Autophagy in human type 2 diabetes pancreatic beta cells. Diabetologia, 2009. 52 (6): p. 1083-6).

### SUMMARY

In one aspect, compounds are provided of the general Formula (I):

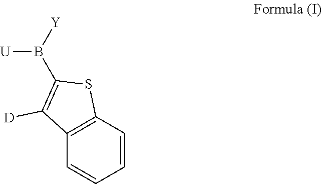

wherein:
D is a functional group selected from a C₁₋ₙ-alkyl, wherein n is an integer from 2 to 5, inclusive, or a substituted alkyl C₁₋ₙ-alkyl -R¹, wherein R¹ is a functional group selected from hydrogen, halo, haloalkyl, cyano, nitro, hydroxyl, alkyl, alkenyl, aryl, alkoxyl, aryloxyl, aralkoxyl, amino, alkylamino, arylamino, aminocarbonyl, carboxyl, or heteroaryl; preferably D is a C₁₋ₙ-alkyl, wherein n is an integer from 2 to 5, inclusive;
B is a linking group selected from a C₂-alkenyl or a C₂-alkynyl, or a substituted C₂-alkenyl-R², wherein R² is a functional group selected from hydrogen, halo, haloalkyl, cyano, nitro, hydroxyl, alkyl, alkenyl, aryl, amino, alkylamino, arylamino and carboxyl; preferably B is a C₂-alkenyl group or a C₂-alkynyl group;
Y is a functional alkylamino group C₁₋ₙ-alkyl-NR³R⁴, wherein n is an integer from 2 to 5, inclusive, wherein R³ and R⁴ are each independently selected from carbonyl, halo, haloalkyl, cyano, nitro, hydroxyl, alkyl, alkenyl, aryl, alkoxyl, amino, alkylamino, arylamino, aminocarbonyl, and carboxyl; preferably R³ is carboxyl and R⁴ is carbonyl;
U may be absent or present, but if present is a functional group selected from hydrogen, a C₁₋ₙ-alkyl, wherein n is an integer from 2 to 5, inclusive, or a substituted alkyl group C₁₋ₙ-alkyl-R⁵, wherein R⁵ is a functional group selected from hydrogen, halo, haloalkyl, cyano, carboxyl, nitro, hydroxyl, alkyl, alkenyl, and aryl; preferably U is hydrogen or absent;
optionally U is absent, and Y and the terminal carbon atom of the B group are forming a closed heterocyclic ring, wherein at least one of the atoms of the ring is nitrogen, and at least one of the atoms of the ring is oxygen; preferably said heterocyclic ring is substituted with one or more groups independently selected from a C₁₋ₙ-alkyl, wherein n is an integer from 2 to 5, inclusive, a substituted alkyl C₁₋ₙ-alkyl-R⁶, wherein n is an integer from 2 to 5, inclusive, and wherein R⁶ is a functional group selected from halo, oxo, haloalkyl, cyano, carbonyl, carboxyl, nitro, hydroxyl, alkyl, alkenyl, aryl; preferably at least two carbon atoms of said heterocyclic ring are substituted, wherein each of the substituents is independently selected from the list provided for R⁶; preferably one of the carbon atoms of said heterocyclic ring is substituted with a C₁₋ₙ-alkyl, wherein n is an integer from 2 to 5, inclusive, and the other carbon atom of said heterocyclic ring is substituted with an oxo (‘═O’) group; or any of its stereoisomers, or any mixture of its stereoisomers, or a pharmaceutically acceptable salt thereof.

Non-limiting examples of compounds of Formula (I) include the compounds of Formula 1, Formula 2 and Formula 3 below.

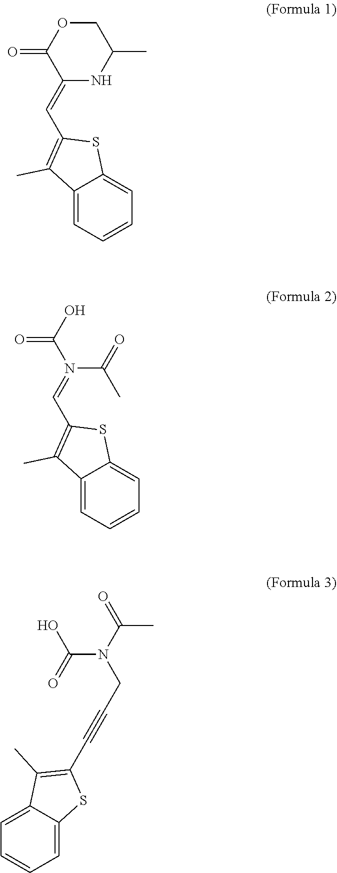

In one aspect, compounds are provided of the general Formula (II):

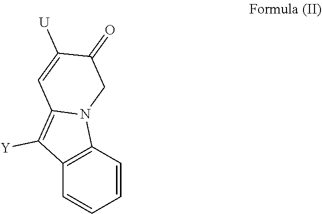

wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁₋ₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

Non-limiting examples of compounds of Formula (II) include the compound of Formula 4 below.

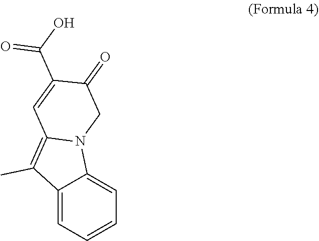 [SMILES: Cc1c2n(c3ccccc13)CC(=O)C(C(=O)O)=C2]

In one aspect, compounds are provided of the general Formula (III):

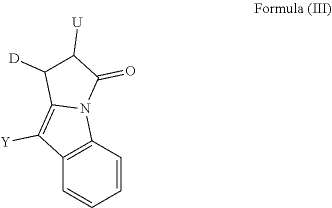

wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁₋ₙ-alkyl, wherein n is an integer from 2 to 5, inclusive;
D is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably D is a C₁₋ₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

Non-limiting examples of compounds of Formula (III) include the compound of Formula 5 below.

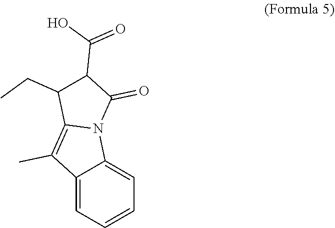 [SMILES: CCC1c2c(C)c3ccccc3n2C(=O)C1C(=O)O]

In one aspect, a pharmaceutical composition containing a compound as described in any of Formulas (I), (II), and (III) is provided together with one or more pharmaceutically acceptable excipients.

In one aspect, a compound is provided according to any one of Formulas (I), (II) or (III) for use in the preparation of a medicament.

In one aspect, use of a compound is provided according to Formulas (I), (II) or (III) for preparing a medicament for treating a disease or disorder associated with impaired mitochondrial clearance or for treating a disease or disorder associated with impaired mitochondrial turnover.

In one aspect, use of a compound is provided according to Formulas (I), (II) or (III) for preparing a medicament for treating a disease or disorder a disease or disorder selected from the list consisting of age-related diseases, age-related disorders, neurodegenerative diseases, neurodegenerative disorders, Alzheimer's disease, Parkinson's disease, Huntington's disease, and Diabetes mellitus type 2.

In one aspect, a method is provided for preventing and/or treating a disease selected from the list consisting of age-related diseases, neurodegenerative disorders, neurodegenerative diseases, neurodegenerative disorders, Alzheimer's disease, Parkinson's disease, Huntington's disease, and Diabetes mellitus type 2, comprising administering to the subject in need thereof a therapeutically effective amount of a compound of any one of Formulae (1), (II), and (III).

In one aspect, a pharmaceutical composition is provided comprising

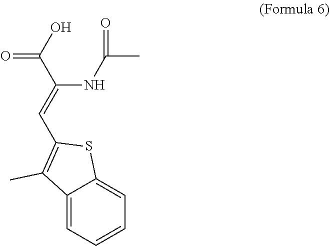 [SMILES: CC(=O)N/C(=C\c1sc2ccccc2c1C)C(=O)O]

or any of its stereoisomers, or any mixture of its stereoisomers, or a pharmaceutically acceptable salt thereof, and a pharmaceutically-acceptable excipient. In some embodiments, the compound of Formula 6 or a pharmaceutical composition thereof if provided in a nanoparticle.

In one aspect, a method is provided for treating a subject having a disease or disorder associated with impaired mitochondrial clearance or having a disease or disorder associated with impaired mitochondrial turnover comprising administering to the subject an effective amount of a pharmaceutical composition comprising the compound of Formula 6.

In one aspect, a method is provided for treating a subject having a disease or disorder selected from the group consisting of age-related diseases, age-related disorders, neurodegenerative diseases, neurodegenerative disorders, Alzheimer's disease, Parkinson's disease, Huntington's disease, and Diabetes mellitus type 2, comprising administering to the subject an effective amount of a pharmaceutical composition comprising the compound of Formula 6.

In one aspect, use is provided for a compound having Formula 6

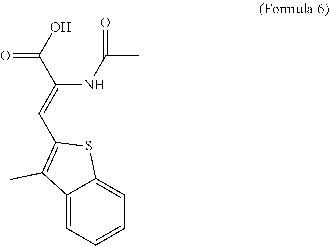 [SMILES: CC(=O)N/C(=C\c1sc2ccccc2c1C)C(=O)O]

or any of its stereoisomers, or any mixture of its stereoisomers, or a pharmaceutically acceptable salt thereof, in the preparation of a medicament for treating a disease or disorder associated with impaired mitochondrial clearance or for treating a disease or disorder associated with impaired mitochondrial turnover.

In one aspect, the use of the compound of Formula 6 is provided, wherein the medicament for treating a disease or disorder a disease or disorder selected from the list consisting of age-related diseases, age-related disorders, neurodegenerative diseases, neurodegenerative disorders, Alzheimer's disease, Parkinson's disease, Huntington's disease, and Diabetes mellitus type 2.

### BRIEF DESCRIPTION OF THE FIGURES

FIGS. 1A-1C demonstrate that Doxycycline impairs oxygen consumption rate and cell growth. INS1 (an insulin secreting cell line) cells (FIGS. 1A and 1B) and primary beta-cells (FIG. 1C) were treated with 1 mg/ml doxycycline for 72 hours. FIG. 1A shows cell growth rate after 24, 48 and 72 hours. FIGS. 1B and 1C show basal Oxygen Consumption Rate (OCR) measured with Seahorse XF 24 respirometer. Average of 4 wells +/−SEM.

FIGS. 2A-2C demonstrates that Rapamycin increases the red-to-green Fluorescence (Fl) and mitochondrial mass in INS1 cells. INS1 cells expressing Mitotimer under EF-1 alpha promoter were treated for 18 hours with rapamycin (100 nM). FIG. 2A shows red-to-green FI. FIGS. 2B and 2C show a Western blot for mitochondrial proteins upon treatment with rapamycin. Note that rapamycin increased mitochondrial mass.

FIG. 3 demonstrates that SPB08007 (the compound of structural Formula 6) and MWP00839 induce mitochondrial turnover in INS1 cells. FIGS. 3A and 3B demonstrate a dose-response in INS1 cells expressing mitoTimer and treated for 18 hours with the tested compounds SPB08007 and MWP00839. The Z-score depicts the standard deviation from the neutral control (DMSO). In FIG. 3C: Mitochondrial mass in INS1 cells treated for 18 hours with either DMSO (control), Urolithin A (25 μM), SPB08007 (“SBP”) (25 μM) or MWP00839 (“MWP”) (11.5 μM) and then stained with Mitotracker green (200 nM). In FIG. 3D: SPB08007 and MWP00839 increase the number of mitochondria inside autolysosomes. INS1 cells expressing mCherry-GFP were exposed to SPB08007, MWP00839, and DMSO for 8 hours and then subjected to confocal microscopy. The area of red fluorescence in the taken images was determined. N=3+/−SEM pANOVA<0.05. FIG. 3E provides representative images. As can be seen, under MWP00839 and SPB08007 an increase in red dots occurs. FIG. 3F provides the Western blot against LC3 in INS1 cells treated with the compounds in presence or absence of bafilomycin (+b) for the last 2 hours. As can be seen, the hits increased LC3II in absence and even more so, in presence of bafilomycin, indicating an increase in autophagic flux.

FIG. 4 demonstrates that MWP00839 is toxic to the cells while SPB08007 helps in recovering from lipotoxicity. In FIGS. 4A and 4B: INS1 cells were treated with either DMSO (control), SPB08007 (25 μM), or MWP00839 (25 μM) for 18 hours and were subjected to an Oxygen consumption rate (OCR) test. Viability test (using XTT staining) was determined after 24 hours exposure. As can be seen, both SPB08007 and MWP00839 decrease OCR, but only MWP00839 displays reduced viability (the control and SPB08007 curves overlap). In FIG. 4C: INS1 cells were treated with 400 μM palmitate (1:5 BSA palmitate) for 24 hours and were then let to recover in presence or absence of SPB08007 (“SBP”) for 18 hours. n=4 (paired t-test).

FIGS. 5A-B depict the MitoTimer model (FIG. 5A) and the screen for identifying hits (FIG. 5B). FIG. 5A shows MitoTimer, translated as a green fluorescent protein, is imported to newly synthesized mitochondria. Over time, the fluorescence shifts from green to red. MitoTimer red/green ratio is an established tool to follow mitochondrial aging and turnover. MitoTimer red/green ratio is directly proportional to the aging of the mitochondria and inversely proportional to mitophagy and mitochondrial biogenesis. FIG. 5B shows a flow chart of the criteria used to establish a given compound as a “hit” which improves mitochondrial clearance.

FIG. 6 depicts the steps used to perform the high content screening for small-molecules which improve mitochondrial clearance based on the Mitotimer model.

FIGS. 7A-E depict the results of high content screening. FIG. 7A shows the final hits number. FIG. 7B shows the chemical structures of the 2 hits identified as mitochondrial turnover stimulators: SPB08007 and MWP00839, from Maybridge library. FIG. 7C shows representative images of Mitotimer fluorescence in INS1 cells treated compound SPB08007 or MWP00839 50 uM for 18 h. Images were acquired with an ImageXpress Micro High-Content Imaging System/Molecular devices microscope. FIGS. 7D and 7E show dose-response effects of compound SPB08007 and MWP00839, respectively, on Mitotimer red/green ratio (expressed as % variation from the DMSO controls).

FIGS. 8A-E show the effect of two hit compounds on mitophagy confirmed by other methods. FIG. 8A shows the co-localization of mCherry-GFP-FIS1101-152 in INS1 cells after 8 h treatment with compounds SPB08007, MWP00839 (25 uM) or DMSO 0.1%. Cells were imaged using a 63× Plan Neofluar objective and the Airyscan module of a Zeiss LSM880 confocal microscope. FIG. 8B shows mitophagy events per cell defined by total area of mCherry-GFP colocalized structures per cell/average area of one mitophagy structure. Data are normalized by DMSO. FIG. 8C shows Mitotracker Green (MTG) Pulse-Chase experiment descriptive scheme—INS1 cells were stained with MTG 500 nM for 30 min, washed and INS1 cells were then treated with compounds SPB08007, MWP00839 (25 uM) or DMSO 0.1%. After 18 h, cells were imaged. As control, cells were loaded with MTG 30 min prior imaging—time “0” (100% MTG staining). Cells were imaged using a 40× lens of a Perkin Elmer Operetta high-content imaging system microscope (FIG. 8D). FIG. 8E shows MTG fluorescence relative to time “0”. Each experiment was repeated three times. For mCherry-GFP-FIS1101-152 assay, 59-85 cells of each group were analyzed in each experiment and for MTG pulse-chase assay, 45 fields were analyzed in each experiment. Values of *p<0.05 vs. DMSO, #p<0.05 vs. “time 0”.

FIGS. 9A-E show that compound SPB08007 increases mitochondrial consumption rates but does not spare capacity. INS1 cells were treated with SPB08007 (25 uM) or DMSO (0.1%) for 48 h. Representative mitochondrial oxygen consumption rates (OCR) of INS1 cells under basal condition (2 mM glucose) and after the subsequent addition of 10 mM glucose, 4 uM oligomicin, 5 uM FCCP and 2 uM antimycin are shown in FIG. 4A. FIG. 9A shows basal mtOCR (last measurement after glucose addition); FIG. 9B shows glucose-stimulated mtOCR (last measurement after glucose addition); FIG. 9C shows ATP-linked mtOCR (glucose-stimulated mtOCR minus oligomycin-insensitive-mtOCR) and FIG. 9D shows maximal mtOCR (highest OCR after FCCP addition), respectively. FIG. 9E shows mitochondrial spare capacity (% of maximum mtOCR relative to basal). OCR values in the presence of antimycin (non-mitochondrial respiration) were subtracted from all quantifications. N=4, *p<0.05 vs. DMSO.

FIGS. 10A-B show that compound SPB08007 increases mitochondrial complex I activity. FIG. 10A shows representative traces of NADH oxidation by mitochondria from INS1 cells previously treated with 25 uM SPB08007 or 0.1% DMSO. FIG. 10B shows complex I activity expressed in OD/min/mg of protein. p=0.0368 vs. DMSO control.

FIGS. 11A-D show that SPB08007 increases mitochondrial membrane potential, without affecting anion radical superoxide formation. INS1 cells were treated for 24 h with SPB08007 (25 uM) or DMSO (0.1%). Mitochondrial membrane potential was analyzed cell by cell through TMRE staining corrected by Mitotracker Green (MTG). Cells were imaged using a 100× Plan Neofluar objective and the Airyscan module of a Zeiss LSM880 confocal microscope. FIG. 11A shows representative images of TMRE and MTG co-localization. FIG. 11B shows TMRE/MTG, relative to DMSO. FIG. 11C shows representative images of Mitosox staining, acquired using a 40× lens of a Perkin Elmer Operetta high-content imaging system microscope; oligomycin (4 uM) was used as positive control. FIG. 11D shows Mitosox Integrated Fluorescence quantification. Each experiment was repeated 2 times, 59-85 cells of each group were analyzed in each experiment for TMRE/MTG, and 48 fields were analyzed in each experiment for Mitosox analysis. *p<0.05 vs. DMSO; ***p<0.05 vs. DMSO and SPB08007.

FIGS. 12A-B show that SPB08007 improves the glucose-stimulated insulin secretion fold in mouse islets. FIG. 12A show mouse islets were pre-incubated with SPB08007 (25 uM) or DMSO (0.1%) for 24 h. Islets were adapted to KRB media containing glucose 2 mM for 1 h, followed by media exchange containing glucose 2 mM (basal) or 12 mM (stimulated). After 1 h, Insulin released in the media was measured by ELISA. FIG. 12B shows insulin secretion fold (glucose stimulated/basal secretion). N=3. *p<0.05 vs. DMSO basal. ***p<0.001 vs. DMSO.

FIGS. 13A-E show that rapamycin as the strongest control of Mitotimer red/green ratio. INS1 cells expressing Mitotimer were treated for 18 h with different known modulators of Mitophagy and/or Autophagy and cells were imaged at an Operetta Perkin Elmer microscope with 40× lens. FIG. 13A shows representative images of cells treated with Urolithin (50 uM), quantified in FIG. 13B. FIG. 13C shows autophagy inhibitors [(Bafolomycin (200 nM), Chloroquine (20 uM)] and Rapamycin (100 nM) effect on Mitotimer red/green ratio. Rapamycin time-response effect on Rapamycin Mitotimer red and green fluorescence: (D) Representative images, (E) Calculation of Mitotimer red/green ratio. *p<0.05 vs. DMSO; **p<0.01 vs. DMSO; ***p<0.001 vs. DMSO.

FIG. 14 depicts an example of a 384 wells plate, imaged for Mitotimer Red and Green Fluorescence after INS1 cells treatment with 10 uM compounds (each well=a compound, DMSO 0.1% and Rapamycin 100 nM as controls) and image analysis on MetaExpress. Data was processed at Gene Data and hits were identified by color, where the strongest shade of blue and red represented the lowest and the highest red:green, respectively. Values in each well are expressed as fold change from DMSO.

FIGS. 15A-D show that SPB08007 reduces overall mitochondrial protein and alters specific mitochondrial protein levels. INS1 cells were treated with SPB08007 (25 (0.1%) for 24 h. FIG. 15A shows representative blots of mitochondrial complexes II, III and I proteins, SDBH, UQCRC2 and NDUFB8, respectively, quantified in FIG. 15B, FIG. 15C and FIG. 15D. Total protein labeling with stain free reagent was used as loading control in all Blots. The quantification is expressed relative to DMSO, control. *p<0.05 vs. DMSO.

FIGS. 16A-B show that SPB08007 does not alter mitochondrial morphology. INS1 cells were treated with SPB08007 (25 uM) or DMSO (0.1%) for 24 h. Mitochondria were stained with Mitotracker Green and TMRE (images represented in FIG. 10). MTG fluorescence was used to calculate Aspect Ratio (AR) (FIG. 16A) and circularity (FIG. 16B) of mitochondrial structures.

FIGS. 17A-B show that SPB08007 reduces levels of ubiquitinated mitochondrial proteins. INS1 cells were treated with SPB08007 (25 uM) or DMSO (0.1%) for 24 h. FIG. 17A: total mitochondrial protein isolated from 4×107 cells were loaded into SDS-PAGE gel previously added of Stain Free reagent, which was used to detect total protein bands in the BioRad Bio Lab gel reader. Protein was transferred into PVDF membrane, blotted to anti-ubiquitin. FIG. 17B: Quantification of total ubiquitin relative to total mitochondrial proteins. The quantification is expressed relative to DMSO, control. *p=0.001.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

The invention relates to use of small organic compounds in stimulating mitochondrial clearance, for the treatment of diseases associated with impaired mitochondrial turnover. Impaired mitochondrial turnover has been reported for a variety of age-associated diseases, including Parkinson disease and Type-2-diabetes. In particular the present invention relates to the compounds of the general Formulae (I), (II) and (III) and (IV), (VIII) and (IX) for the treatment of diseases associated with impaired mitochondrial turnover, such as for example age-associated diseases, including, without being limited to, Parkinson disease, Huntington's disease, Alzheimer's disease, dementia and Type-2-diabetes. The present invention also embraces pharmaceutical compositions comprising these compounds and methods of using the compounds and their pharmaceutical compositions.

It has now been found by the present inventors that members of a novel group of benzothiophene-based compounds stimulate mitochondrial clearance, without inducing toxicity, and are thus useful as stimulators of mitochondrial turnover.

The present invention provides substituted benzothiophene-derivatives of general Formula (I)

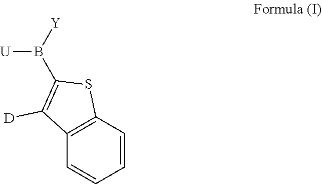

wherein the groups B, Y, U and D are as defined hereinafter, any of its stereoisomers, or any mixture of its stereoisomers, or a pharmaceutically acceptable salt thereof.

Compounds of general Formulae (I), (II) and (III), and (IV), (VIII) and (IX) as defined hereinafter, are suitable for stimulating mitochondrial clearance. Compounds of general Formulae (I), (II) and (III) and (IV), (VIII) and (IX), are also suitable for stimulating mitochondrial turnover.

The invention is further directed to pharmaceutical compositions containing a compound of Formulae (I), (II) or (III) and (IV), (VIII) and (IX) according to the invention, as well as to the use of the compounds of Formulae (I), (II) and/or (III) and/or (IV), (VIII) and (IX) for preparing a pharmaceutical composition for the treatment of diseases and/or disorders, especially diseases and/or disorders associated with impaired mitochondrial turnover.

Other aspects and embodiments of the present invention will become apparent to the skilled person from the following description.

According to one aspect of the invention there is provided a compound of the general Formula (I):

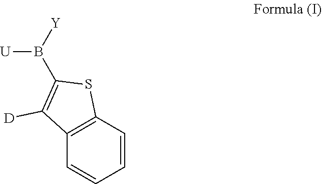

wherein:
D is a functional group selected from a C₁₋ₙ-alkyl, wherein n is an integer from 2 to 5, inclusive, or a substituted alkyl C₁₋ₙ-alkyl -R¹, wherein R¹ is a functional group selected from hydrogen, halo, haloalkyl, cyano, nitro, hydroxyl, alkyl, alkenyl, aryl, alkoxyl, aryloxyl, aralkoxyl, amino, alkylamino, arylamino, aminocarbonyl, carboxyl, or heteroaryl; preferably D is a C₁₋ₙ-alkyl, wherein n is an integer from 2 to 5, inclusive, in some embodiments D is a C₁ -alkyl;
B is a linking group selected from a C₂-alkenyl or a C₂-alkynyl, or a substituted C₂-alkenyl-R², wherein R² is a functional group selected from hydrogen, halo, haloalkyl, cyano, nitro, hydroxyl, alkyl, alkenyl, aryl, amino, alkylamino, arylamino and carboxyl; preferably B is a C₂-alkenyl group or a C₂-alkynyl group;
Y is a functional alkylamino group C₁₋ₙ-alkyl-NR³R⁴, wherein n is an integer from 2 to 5, inclusive, wherein R³ and R⁴ are each independently selected from carbonyl, halo, haloalkyl, cyano, nitro, hydroxyl, alkyl, alkenyl, aryl, alkoxyl, amino, alkylamino, arylamino, aminocarbonyl, and carboxyl; preferably R³ is carboxyl and R⁴ is carbonyl;
U may be absent or present, but if present is a functional group selected from hydrogen, a C₁₋ₙ-alkyl, wherein n is an integer from 2 to 5, inclusive, or a substituted C₁₋ₙ-alkyl-R⁵, wherein R⁵ is a functional group selected from hydrogen, halo, haloalkyl, cyano, carboxyl, nitro, hydroxyl, alkyl, alkenyl, and aryl; preferably U is hydrogen or absent;
optionally U is absent, and Y and the terminal carbon atom of the B group are forming a closed heterocyclic ring, wherein at least one of the atoms of the ring is nitrogen, and at least one of the atoms of the ring is oxygen; preferably said heterocyclic ring is substituted with one or more groups independently selected from a C₁₋ₙ-alkyl, wherein n is an integer from 2 to 5, inclusive, a substituted C₁₋ₙ-alkyl-R⁶, wherein n is an integer from 2 to 5, inclusive, and wherein R⁶ is a functional group selected from halo, oxo, haloalkyl, cyano, carbonyl, carboxyl, nitro, hydroxyl, alkyl, alkenyl, aryl; preferably at least two carbon atoms of said heterocyclic ring are substituted, wherein each of the substituents is independently selected from the list provided for R⁶; preferably one of the carbon atoms of said heterocyclic ring is substituted with a C₁₋ₙ-alkyl, wherein n is an integer from 2 to 5, inclusive, and another carbon atom of said heterocyclic ring is substituted with an oxo (‘═O’) group.

In one embodiment of a compound of general Formula (I), D is a C₁-alkyl, B is a C₂-alkenyl group, Y is an alkylamino group C₁-alkyl-NR³R⁴, wherein R³ is carboxyl and R⁴ is carbonyl, and U is hydrogen.

In another embodiment of a compound of general Formula (I), D is a C₁-alkyl, B is a C₂-alkynyl group, Y is an alkylamino group C₁-alkyl-NR³R⁴, wherein R³ is carboxyl and R⁴ is carbonyl, and U is absent.

In yet another embodiment of a compound of general Formula (I), D is a C₁-alkyl, B is a C₂-alkenyl group, U is absent, and Y and the terminal carbon atom of the B group are forming a closed heterocyclic ring, wherein one of the atoms of the ring is nitrogen, and one of the atoms of the ring is oxygen; said heterocyclic ring is substituted at two carbon atoms, wherein one carbon atom is substituted with a C₁₋ₙ-alkyl, wherein n is an integer from 2 to 5, inclusive, and the other carbon atom is substituted with an oxo (‘═O’) group.

The invention also relates to the stereoisomers, mixtures thereof, and salts, particularly the physiologically acceptable salts, of the compounds of general Formula (I) according to the invention.

According to one aspect of the invention there is provided a compound of the general Formula (IV):

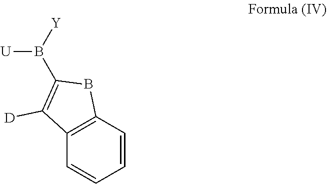

wherein:
D is a C₁₋ₙ-alkyl, or a C₁₋ₙ-alkyl substituted with R¹, wherein R¹ is selected from hydrogen, halo, haloalkyl, cyano, nitro, hydroxyl, alkyl, alkenyl, aryl, alkoxyl, aryloxyl, aralkoxyl, amino, alkylamino, arylamino, aminocarbonyl, carboxyl, or heteroaryl;
B is a linking group selected from a C₂-alkenyl or a C₂-alkynyl, or a C₂-alkenyl substituted with R², wherein R² is selected from hydrogen, halo, haloalkyl, cyano, nitro, hydroxyl, alkyl, alkenyl, aryl, amino, alkylamino, arylamino and carboxyl;
Y is C₁₋ₙ-alkyl-NR³R⁴, wherein R³ and R⁴ are each independently selected from carbonyl, alkylcarbonyl, halo, haloalkyl, cyano, nitro, hydroxyl, alkyl, alkenyl, aryl, alkoxyl, amino, alkylamino, arylamino, aminocarbonyl, and carboxyl;
U may be absent or present; when present, U is a functional group selected from hydrogen, a C₁₋ₙ-alkyl, or a C₁₋ₙ-alkyl substituted with R⁵, wherein R⁵ is selected from hydrogen, halo, haloalkyl, cyano, carboxyl, nitro, hydroxyl, alkyl, alkenyl, and aryl;
when U is absent, Y and the terminal carbon atom of the B group to which Y is attached are optionally forming a heterocyclic ring, wherein at least one of the atoms of the ring is nitrogen, and at least one of the atoms of the ring is oxygen;
n is an integer from 2 to 5;
or any of its stereoisomers, or any mixture of its stereoisomers, or a pharmaceutically acceptable salt thereof.

According to one aspect of the invention, there is provided a compound of Formula (IV), wherein the compound is represented by a compound of Formula (V)

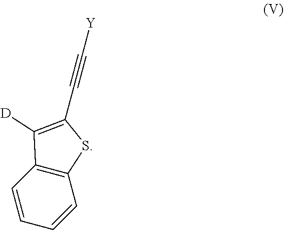

According to one aspect of the invention, there is provided a compound of Formula (IV), wherein said compound is represented by a compound of Formula (VI) or (VII)

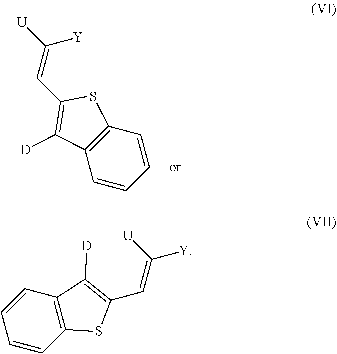

In some embodiments, a compound is provided according to Formula (IV), wherein D is a C₁₋ₙ-alkyl.

In some embodiments, a compound is provided according to Formula (IV), wherein B is a C₂-alkenyl group or a C₂-alkynyl group.

In some embodiments, a compound is provided according to Formula (IV), wherein R³ is carboxyl and R⁴ is carbonyl.

In some embodiments, a compound is provided according to Formula (IV), wherein U is hydrogen or absent.

In some embodiments, a compound is provided according to Formula (IV), where the formed heterocyclic ring is substituted with one or more groups independently selected from a C₁₋ₙ-alkyl or a C₁₋ₙ-alkyl substituted with R⁶, and wherein R⁶ is selected from halo, oxo, haloalkyl, cyano, carbonyl, carboxyl, nitro, hydroxyl, alkyl, alkenyl, and aryl.

In some embodiments, a compound is provided according to Formula (IV), wherein at least two carbon atoms of said formed heterocyclic ring are substituted with R⁶, wherein R⁶ is independently selected from halo, oxo, haloalkyl, cyano, carbonyl, carboxyl, nitro, hydroxyl, alkyl, alkenyl, and aryl.

In some embodiments, a compound is provided according to Formula (IV), wherein one of the carbon atoms of said formed heterocyclic ring is substituted with a C₁₋ₙ-alkyl, and the other carbon atom of said formed heterocyclic ring is substituted with an oxo (‘═O’) group.

According to another aspect of the invention there is provided a compound of the general Formula (II):

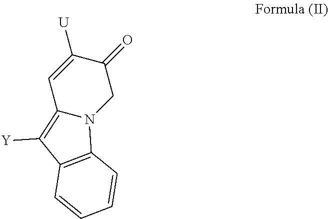

wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁-alkyl or a Cₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

In one embodiment of a compound of general Formula (II), Y is C₁-alkyl and U is a carboxyl group.

According to one aspect of the invention there is provided a compound of the general Formula (VIII):

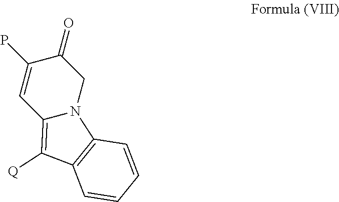

wherein:
Q is selected from a C₁₋ₙ-alkyl, or a C₁₋ₙ-alkyl substituted with R¹, wherein R¹ is selected from hydrogen, halo, haloalkyl, cyano, nitro, hydroxyl, alkyl, alkenyl, aryl, amino, alkylamino, arylamino, aminocarbonyl, carboxyl, and heteroaryl;
P is selected from carbonyl, carboxyl, halo, haloalkyl, cyano, nitro, hydroxyl, alkyl, alkenyl, aryl, alkoxyl, amino, alkylamino, arylamino, and aminocarbonyl;
n is an integer from 2 to 5;
or any of its stereoisomers, or any mixture of its stereoisomers, or a pharmaceutically acceptable salt thereof.

In some embodiments, a compound is provided according to Formula (VIII), wherein Q is a C₁₋ₙ-alkyl.

In some embodiments, a compound is provided according to Formula (VIII), wherein P is carboxyl.

According to another aspect of the invention there is provided a compound of the general Formula (III):

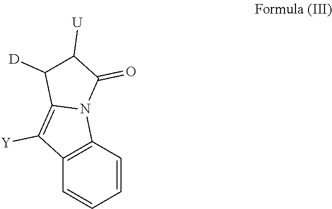

wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁-alkyl or a Cₙ-alkyl, wherein n is an integer from 2 to 5, inclusive;
D is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably D is a C₁-alkyl or a Cₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

In one embodiment of a compound of general Formula (III), Y is C₁-alkyl, D is a C₂-alkyl and U is a carboxyl group.

According to one aspect of the invention there is provided a compound of the general Formula (IX):

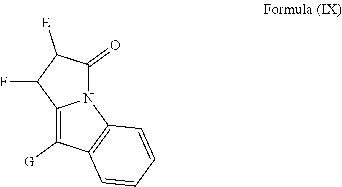

wherein:
G is selected from a C₁₋ₙ-alkyl, or a C₁₋ₙ-alkyl substituted with R¹, wherein R¹ is selected from hydrogen, halo, haloalkyl, cyano, nitro, hydroxyl, alkyl, alkenyl, aryl, amino, alkylamino, arylamino, aminocarbonyl, carboxyl, and heteroaryl;
F is selected from a C₁₋ₙ-alkyl, or a C₁₋ₙ-alkyl substituted with R¹, wherein R¹ is selected from hydrogen, halo, haloalkyl, cyano, nitro, hydroxyl, alkyl, alkenyl, aryl, amino, alkylamino, arylamino, aminocarbonyl, carboxyl, and heteroaryl;
E is selected from carbonyl, carboxyl, halo, haloalkyl, cyano, nitro, hydroxyl, alkyl, alkenyl, aryl, alkoxyl, amino, alkylamino, arylamino, and aminocarbonyl;
n is an integer from 2 to 5;
or any of its stereoisomers, or any mixture of its stereoisomers, or a pharmaceutically acceptable salt thereof.

In some embodiments, a compound is provided according to Formula (IX), wherein G is a C₁₋ₙ-alkyl.

In some embodiments, a compound is provided according to Formula (IX), wherein F is a C₁₋ₙ-alkyl.

In some embodiments, a compound is provided according to Formula (IX), wherein E is carboxyl.

The invention also relates to the stereoisomers, mixtures thereof and salts thereof, particularly the physiologically acceptable salts, of the compounds of general Formulae (I), (II) and (III), according to the invention.

Table 1 provides non-limiting examples of compounds of general Formulae (I), (II) and (III), and (IV), (VIII) and (IX). It includes compounds as follows: compound of structural Formula 1, compound of structural Formula 2, compound of structural Formula 3, compound of structural Formula 4 and compound of structural Formula 5.

TABLE 1

| Formula No. | Structure | Also identified herein as: |
|---|---|---|
| 1 | 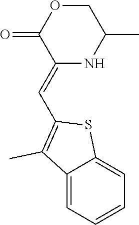 [SMILES: Cc1c(/C=C2\NC(C)COC2=O)sc2ccccc12] | GL-21 |
| 2 | 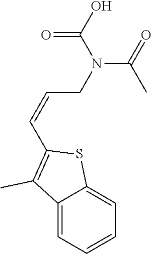 [SMILES: CC(=O)N(C/C=C\c1sc2ccccc2c1C)C(=O)O] | GL-24 |
| 3 | 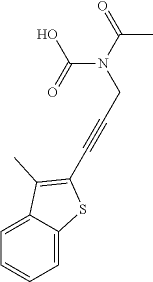 [SMILES: CC(=O)N(CC#Cc1sc2ccccc2c1C)C(=O)O] | GL-25 |
| 4 | 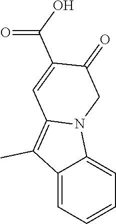 [SMILES: Cc1c2n(c3ccccc13)CC(=O)C(C(=O)O)=C2] | GL-26 |
| 5 | 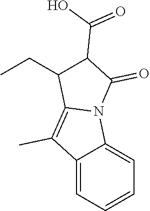 [SMILES: CCC1c2c(C)c3ccccc3n2C(=O)C1C(=O)O] | GL-30r |

According to another aspect of the invention, provided herein is a process for the preparation of a compound of general Formula (I), (II), (III), (IV), (VIII) and (IX). The compounds of general Formula (I), (II), (III), (IV), (VIII) and (IX) according to the invention may be obtained using known methods of synthesis.

The invention also relates to the stereoisomers, mixtures thereof and salts, particularly the physiologically acceptable salts, of the compounds of structural formulae 1, 2, 3, 4 and 5 (as provided in Table 1 herein).

The compounds of general Formulae (I), (II), (III), (IV), (VIII) and (IX) or intermediate products in the synthesis of compounds of general Formulae (I), (II), (III), and (IV), (VIII) and (IX) may be resolved into their stereoisomers on the basis of their physical-chemical differences using methods known in the art. For example, cis/trans mixtures may be resolved into their cis and trans isomers by chromatography. For example, enantiomers may be separated by chromatography on chiral phases or by recrystallisation from an optically active solvent or by enantiomer-enriched seeding.

The compounds of general Formulae (I), (II), (III), (IV), (VIII) and (IX) and the compounds of structural formulae 1, 2, 3, 4, 5 and 6 may be converted into the salts thereof, particularly physiologically acceptable salts for pharmaceutical use.

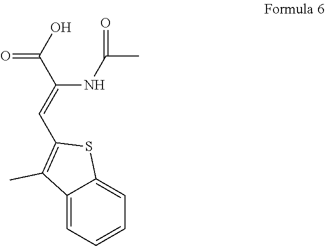

The compound of structural Formula 6 is also identified herein as SPB08007.

Suitable salts of the compounds of general Formulae (I), (II), (III), (IV), (VIII) and (IX) and of the compounds of structural formulae 1, 2, 3, 4, 5 and 6, may be formed with organic or inorganic acids, such as, without being limited to hydrochloric acid, hydrobromic acid, sulphuric acid, phosphoric acid, lactic acid, acetic acid, succinic acid, citric acid, palmitic acid or maleic acid. Compounds of general Formulae (I), (II), (III), (IV), (VIII) and (IX) containing a carboxy group, may be converted into the salts thereof, particularly into physiologically acceptable salts for pharmaceutical use, with organic or inorganic bases. Suitable bases for this purpose include, for example, sodium hydroxide, potassium hydroxide, ammonium hydroxide, arginine or ethanolamine

According to another aspect provided herein are uses of the compounds of general Formulae (I), (II), (III), (IV), (VIII) and (IX) such as, without being limited to, the compounds of structural formulae 1, 2, 3, 4 and 5, and of the compound of structural formula 6, for example as stimulators of mitochondrial turnover.

Another aspect of the invention relates to the use of compound according to general Formulae (I), (II), (III), (IV), (VIII) and (IX) such as, without being limited to, the compounds of structural Formulae 1, 2, 3, 4 and 5, and of the compound of structural formula 6, in the preparation of medicaments for treatment of diseases as described herein.

The compounds of general Formulae (I), (II), (III), (IV), (VIII) and (IX) especially the specific compounds of Formulae 1, 2, 3, 4 and 5, and the compound of structural formula 6 are modulators of mitochondrial turnover. The effect of the compounds of the invention and of the specific compounds of Formulae 1, 2, 3, 4, 5 and 6 on mitochondrial turnover, i.e. their ability to stimulate mitochondrial clearance, is determined by “MitoTimer”, a green fluorescent protein targeted to the mitochondrial matrix which emission shifts to red within about 18 hours after its translation. The ratio of the red-to-green fluorescence intensity (FI) was shown to be a reliable parameter of the rate of mitochondrial turnover (Ferree, A. W., et al., MitoTimer probe reveals the impact of autophagy, fusion, and motility on subcellular distribution of young and old mitochondrial protein and on relative mitochondrial protein age. Autophagy, 2013. 9 (11): p. 1887-96; Trudeau, K. M., et al., Measurement of mitochondrial turnover and life cycle using MitoTimer. Methods Enzymol, 2014. 547: p. 21-38); and according to other methods as described herein.

The compounds of general Formulae (I), (II), (III), (IV), (VIII) and (IX) have IC50 values for several activities in the range from 1.6 μM to 50 uM.

In view of the ability of the compounds that are described herein to stimulate mitochondrial clearance, the compounds of general Formulae (I), (II), (III), (IV), (VIII) and (IX) especially the specific compounds of Formulae 1, 2, 3, 4 and 5, and the compound of structural formula 6, and the pharmaceutically acceptable salts thereof, may be suitable for treating and/or preventing all those conditions or diseases that can be influenced by enhanced mitochondrial turnover. Therefore the compounds according to general Formulae (I), (II), (III), (IV), (VIII) and (IX) especially the each of the specific compounds of Formulae 1, 2, 3, 4 and 5, and the compound of structural formula 6, and the pharmaceutically acceptable salts thereof, are particularly suitable for the prevention or treatment of diseases or conditions associated with impaired mitochondrial turnover, such as, without being limited to, age-related diseases and/or disorders, and neurodegenerative diseases and/or disorders. The compounds of general Formulae (I), (II), (III), (IV), (VIII) and (IX)especially the specific compounds of Formulae 1, 2, 3, 4 and 5, and the compound of structural formula 6, and the pharmaceutically acceptable salts thereof, are also particularly suitable for the prevention or treatment of diseases or conditions such as Alzheimer's disease, Parkinson's disease, Huntington's disease, Diabetes mellitus type 2 and related diseases and/or disorders.

The compounds of general Formulae (I), (II), (III), (IV), (VIII) and (IX), such as, without being limited to, the compounds of structural formulae 1, 2, 3, 4 and 5, and the compound of structural formula 6, and the pharmaceutically acceptable salts thereof, may be formulated in a pharmaceutical composition, optionally comprising other active substances, and one or more of inert conventional excipients, as known to the skilled artisans. The pharmaceutical compositions may be prepared according to the general guidance provided in the art, e.g. by Remington, The Science and Practice of Pharmacy (formerly known as Remington's Pharmaceutical Sciences), ISBN 978-0-85711-062-6. The pharmaceutical compositions, e.g. in the form solid dosage forms, topical dosage form, and/or parenteral dosage forms, e.g. tablets, capsules, creams, ointments, patches, injections, and others as known in the art constitute another aspect of the invention.

The compounds of general Formulae (I), (II), (III), (IV), (VIII) and (IX), particularly of structural formulae 1, 2, 3, 4 and 5, and the compound of structural formula 6, and the pharmaceutically acceptable salts thereof, may be encapsulated, for example, formulated as nanoparticles. The nanoparticles may be prepared in well-known polymers, e.g. polylactic-co-glycolic acid, e.g., as described in H. K. Makadia, S. J. Siegel, Poly Lactic-co-Glycolic Acid (PLGA) as Biodegradable Controlled Drug Delivery Carrier, Polymers (Basel), 3 (2011) 1377-1397; and others. Generally, the compounds may be co-dissolved with the polymer in a suitable organic solvent, and the organic phase may be then dispersed in an aqueous phase comprising stabilizers and/or surface active agents. The stabilizers may be, e.g. polyvinyl alcohol, with molecular weights from about 89000 to 98000, and hydrolysis degree from about 99%. Upon evaporation of organic solvent from the aqueous phase, the nanoparticles may be purified, e.g. by centrifugation and washing. Other method for preparing and using nanoparticles are described in Patra et al., Nano based drug delivery systems: recent developments and future prospects, Journal of Nanobiotechnology (2018) 16:71; and Rizvi et al., Applications of nanoparticle systems in drug delivery technology, Saudi Pharm J. (2018) 26 (1):64-70.

The encapsulated compounds, e.g. in form of nanoparticles, of general Formulae (I), (II), (III), (IV), (VIII) and (IX), such as, without being limited to, the compounds of structural formulae 1, 2, 3, 4 and 5, and the compound of structural formula 6, and the pharmaceutically acceptable salts thereof, may be advantageously used in various routes of administration. Intranasal route may be suitable mode of administration in this regard. Alternatively, the nanoparticles may be administered systemically.

The dose of compounds of general Formulae (I), (II), (III), (IV), (VIII) and (IX), such as, without being limited to, the compounds of structural formulae 1, 2, 3, 4 and 5, and the compound of structural formula 6, and the pharmaceutically acceptable salts thereof, required to achieve treatment or prevention of a disease or a disorder or a condition usually depends on the pharmacokinetic and pharmacodynamic properties of the compound which is to be administered, the patient, the nature of the disease, disorder or condition and the method and frequency of administration.

Suitable dosage ranges for compounds of general Formulae (I), (II), (III), (IV), (VIII) and (IX), such as, without being limited to, the compounds of structural formulae 1, 2, 3, 4 and 5, and the compound of structural formula 6, and the pharmaceutically acceptable salts thereof, may be from 1.0 to 100 mg/kg body weight.

Accordingly, in another aspect there is provided a method for preventing or treating a disease selected from the list consisting of age-related diseases and disorders, neurodegenerative diseases and/or disorders, Alzheimer's disease, Parkinson's disease, Huntington's disease, and Diabetes mellitus type 2, comprising administering to the subject in need thereof a therapeutically effective amount of a compound of general Formulae (I), (II), (III), (IV), (VIII) and (IX), as defined herein, such as, without being limited to, the compounds of structural Formulae 1, 2, 3, 4 and 5, and the compound of structural Formula 6.

Mitochondrial Quality Control (mtQC) reflects all cellular processes that ensure the maintenance of the pool of healthy and functional mitochondria, which is critical to meet the cellular demands of energy, calcium buffering, metabolic flux and control of apoptosis. At one end of mtQC, there is the synthesis of new mitochondria through biogenesis, and at the other end, their elimination through selective autophagy, mitophagy.

The balance between these two opposing forces determines the mitochondrial turnover rate, allowing for a constant, tissue-specific, mitochondrial mass, which can be challenged by physiological demands. Physical exercise and calorie restriction, for example, enhance mitochondrial biogenesis, increasing net mitochondrial volume, while mitophagy is triggered in some cell types during developmental stages, in response to oxygen deprivation and as a consequence of mitochondrial damage.

During aging, there is a decline in mitochondriogenesis and mitophagy. While reduced biogenesis compromises the energy availability for the cell, reduced ability to eliminate dysfunctional mitochondria has deleterious effects, since these organelles have declined dynamics and trafficking and are more prone to generate reactive oxygen species (ROS). Oxidative damage to biomolecules is largely related to aging and cell death. Long-lived cells such as neurons, cardiomyocytes and beta-cells exhibit very low or non-existent replication capacity, and are as such particularly vulnerable to autophagy and mitophagy impairment and to excessive mitochondrial ROS formation.

Boosting autophagy has been shown to be beneficial in animal models of neurodegenerative and cardiac diseases, hepatic fibrosis, diabetes, and cancer, and small-molecule enhancers of autophagy are increasingly being tested with promising results. General stimulation of autophagy may help to revert mitophagy impairment when the impairment results from a compromise in the autophagic machinery (lysosome/autophagosome axis). However, stimulating autophagy per se does not address mitophagy-specific pathways. It is widely accepted that mitophagy is a selective event, with its own regulators, and mitophagy-targeted tools for high-throughput screening are being pursued.

To date, genome-wide screens based upon the activation of Pink1/Parkin were developed to better understand activators of this regulatory axis. However, a broader assay for mitochondrial turnover is required to identify small molecules with different targets, and potentially unveil new mitophagy regulatory pathways, in a high-throughput manner While Pink1 and Parkin are strongly implicated in stress-induced mitophagy, very few studies have focused on physiological mitophagy and fewer on basal mitophagy.

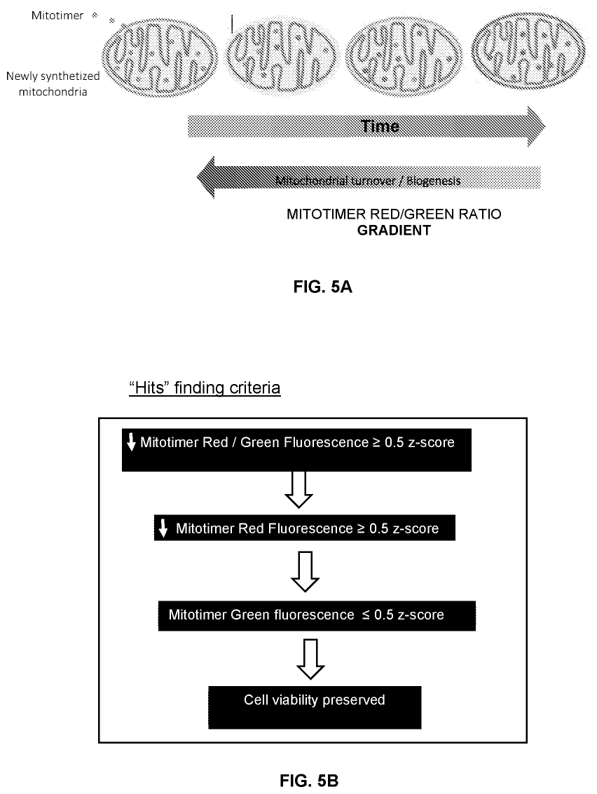

MitoTimer was previously engineered, by adding a mitochondrial-targeting sequence to the Timer protein (Hernandez, G., Thornton, C., Stotland, A., Lui, D., Sin, J., Ramil, J., et al. (2013) MitoTimer: a novel tool for monitoring mitochondrial turnover. Autophagy 9, 1852-1861; Ferree, A. W., Trudeau, K., Zik, E., Benador, I. Y., Twig, G., Gottlieb, R. A., et al. (2013) MitoTimer probe reveals the impact of autophagy, fusion, and motility on subcellular distribution of young and old mitochondrial protein and on relative mitochondrial protein age. Autophagy. 9, 1887-1896), a mutant of Ds-Red, which emits green fluorescence when newly translated, with the emission shifting over to red, a property which allows MitoTimer to be used to follow mitochondrial turnover (FIG. 5), as demonstrated by us and others.

Screening 15,000 small molecules in immortalized beta-cells INS1 stably expressing MitoTimer led to the discovery of two chemically-related benzothiophene derivatives (SPB08007 and MWP00839) that were able to increase basal mitochondrial turnover through enhanced mitophagy without triggering loss of mitochondrial membrane potential (ψm). Enhanced turnover promoted by SPB08007 resulted in a pool of healthier mitochondria with improved mitochondrial function, Complex I activity, and higher Complex II and III content. In mouse islets, insulin secretion capacity was enhanced due to lower basal secretion, an index associated with islet health.

All patents, patent applications, and scientific publications cited herein are hereby incorporated by reference in their entirety.

The following examples are presented in order to more fully illustrate the preferred embodiments of the invention. It should in no way be construed, however, as limiting the broad scope of the invention.

### EXAMPLES

### Example 1. Generation of a New MitoTimer Lentiviral Plasmid

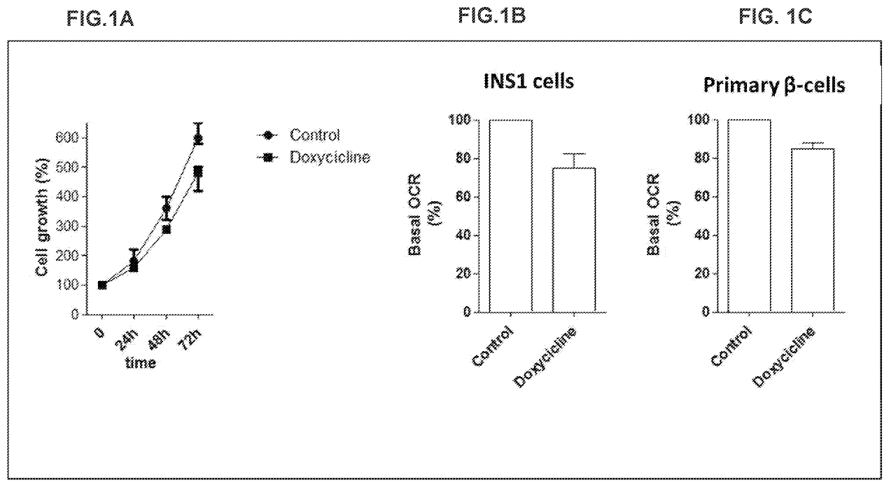

The original MitoTimer plasmid consists of a lentiviral plasmid in which the MitoTimer sequence is placed downstream to a Tet-on promoter (i.e. doxycycline-dependent). Doxycycline was however recently reported to impair mitochondrial function in certain cell types (Moullan, N., et al., Tetracyclines Disturb Mitochondrial Function across Eukaryotic Models: A Call for Caution in Biomedical Research. Cell Rep, 2015). Preliminary tests showed that in INS1 cells (an insulin secreting cell line) it affects respiration as well. The results are shown in FIG. 1. We therefore generated a new MitoTimer lentiviral plasmid which is regulated by EF1α, a constitutively active promoter. The lenti-vector for MitoTimer was constructed using conventional restriction and ligation. Briefly, MitoTimer cassette was amplified by PCR with primers having flanking restriction sites. Amplified fragment was purified, and digested by NotI and BglII. This fragment was ligated into the first ORF of pHAGE2 lenti-vector, having a constitutive EF1α promoter, and followed by an IRES-PURO cassette enabling positive selection. Lentiviral particles were generated in 293T cells by transfection of the pHAGE2 plasmid together with 4 packaging plasmids (namely Rev1, Tat1, Hgpm2, and VSVG). Supernatants were collected over 4 days and concentrated by ultracentrifugation. Aliquots of concentrated lentiviral particles were stored at −80° C. The vector enables transduction of cell-lines and the selection for positive expressing cells by puromycin.

INS1 cells were stably transduced with MitoTimer by lentiviral infection and were tested using known modulators of autophagy. The paradigm used was based on previous reports showing that the shift in emission from green to red occurs within 18 hours. Assuming that this shift is constant, changes in red FI occurring during this frame of time can result from an effect on MitoTimer clearance and not from an effect on synthesis. The paradigm employed consisted therefore of a treatment lasting 18 hours followed by imaging.

### Example 2. Testing of the New MitoTimer Lentiviral Plasmid

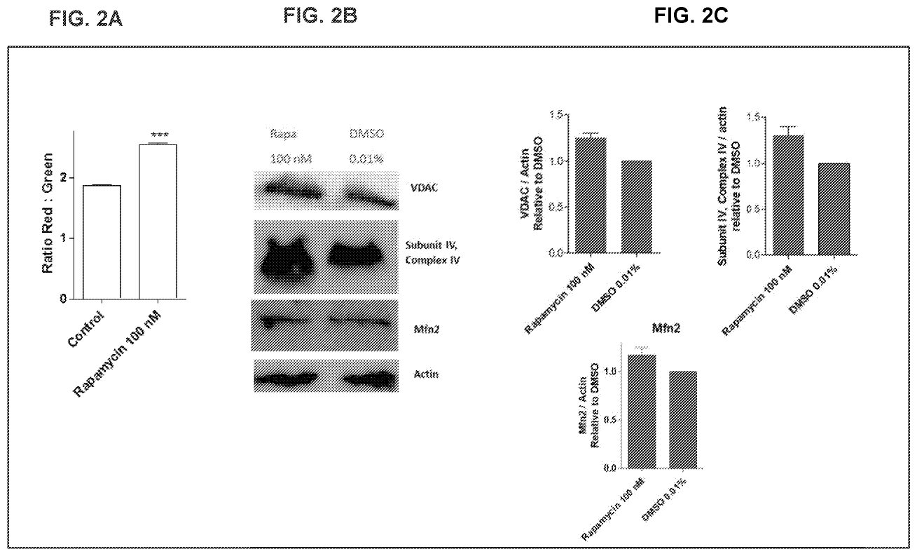

The new MitoTimer plasmid described in Example 1 herein was tested using a series of molecules known to modulate either autophagy or mitochondrial function. None of those molecules showed any reduction in red-to-green FI (data not shown), suggesting that mitochondrial clearance is already elevated under homeostasis in INS1 cells. Surprisingly, treatment with rapamycin, a bona fide stimulator of autophagy, led to an increase in red-to-green FI. This effect was highly reproducible, as shown in FIG. 2A, and was the result of increase in red FI, indicating a reduction of clearance. To ensure that rapamycin indeed suppresses mitochondrial clearance, we examined its effect on mitochondrial mass. Eighteen hours of exposure to rapamycin resulted in an increase in VDAC, Mfn2 and complex IV indicating an increase in mass. The results are provided in FIGS. 2B and 2C. It is possible that this apparent discrepancy is due to the peculiarity of beta-cells, which under autophagic stress (starvation), increase mitochondrial fusion, which prevents mitophagy (Gomes, L. C., et al., During autophagy mitochondria elongate, are spared from degradation and sustain cell viability. Nat Cell Biol, 2011. 13 (5): p. 589-98).

### Example 3. Screening Method to Identify Compounds that Enhance Mitochondrial Clearance/Turnover

The ability of the compounds of the invention to stimulate mitochondrial clearance/turnover is tested by the following screening method.

The filters used for the screen relied on the standard deviation relative to the neutral control and are provided in Table 2.

TABLE 2

| Index | Column | Condition | | |
|---|---|---|---|---|
| 1 | Red Green Ratio:Activity | Outside range | −3 > R/G ratio > | 10.34 |
| | | [−3:10.34] | | |
| 2 | Red Intensity:Activity | Outside range | −5.1 > Red Intensity > | 4.5 |
| | | [−5.144:4.562] | | |
| 3 | Cell Count | >−5.978 | Cell number > | −5.9 |
| 4 | Red Green Ratio:Condensing | >0.1901 | pValue > | 0.2 |
| | ‘Mean’:p-Value | | | |
| 5 | Green Intensity:Activity | <15 | Green Intensity < | 15 |

Stimulators of mitochondrial clearance were defined as those inducing a red-to-green FI below −3 and a red intensity below −5.1. Suppressors of mitochondrial clearance were defined as those inducing a red-to-green FI above 10.34 and a red intensity above 4.5. Highly toxic compounds were discarded by picking only compounds that left a higher cell number than the −5.9. Compounds displaying high green FI (above 15) were also discarded to prevent the inclusion of autofluorescent molecules. Only molecules that filled the above criteria in both duplicates were further tested.

The screen was performed during 8 sessions in Weizmann institute. All compounds were tested at a concentration of 10 μM, in duplicates. The images were processed to reduce background (using Metamorph software) and then analyzed in GeneData software.

The screen was performed using three libraries: (1) a library consisting of 1,280 pharmacologically active compounds (LOPAC); (2) a library consisting of 3,840 compounds consisting of highly purified plant metabolites (Analyticon); (3) a diversity library consisting of 9,920 compounds (MayBridge). Using the above filters we detected 128 Hits (0.93%): 78 stimulators and 50 inhibitors. We subjected those compounds to a dose-response assay (concentrations: 10, 3.33, 1.11, 0.37 and 0.12 μM). Autofluorescence was determined by administrating the compounds to cells that do not express MitoTimer. The vast majority of compounds did not display any autofluorescence (Data not shown). Only compounds that displayed a dose-response curve reproduced in 3 separate experiments without autofluorescence were further examined.

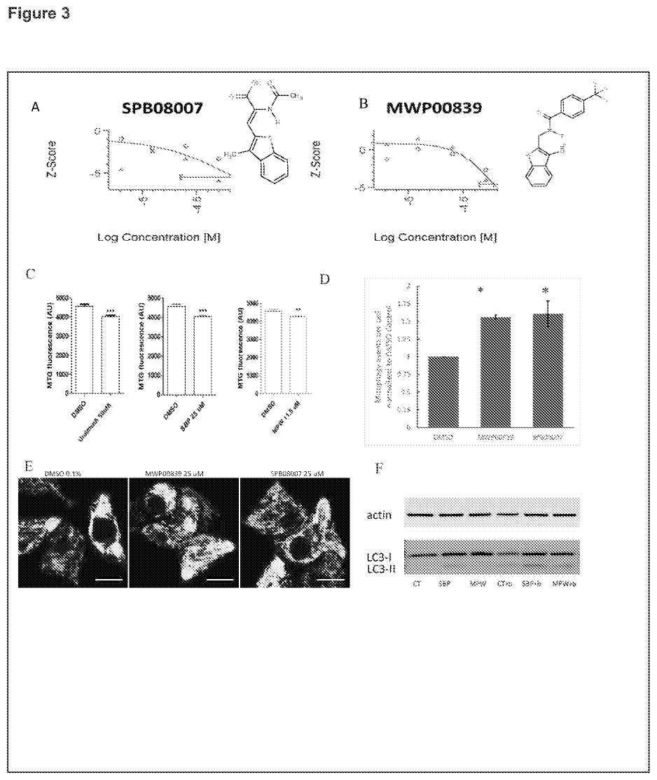

Among the stimulators, only two (both from MayBridge) showed a clear dose-response: SPB08007 (compound of structural Formula 6) and MWP00839. The results are provided in FIGS. 3A and 3B), with SPB08007 displaying an activity of −3.01837 and a qEC50 of 0.000049 and MWP00839 displaying an activity of −0.7122 and a qEC50 of 0.0000497. The optimal dose for stimulating a reduction in red-to-green FI was 25 μM for both molecules. In view of the increase in mitochondrial clearance, we anticipated a reduction in mitochondrial mass. Staining of INS1 cells treated with the compounds and stained with mitotracker green (a fluorescent probe that binds mitochondrial proteins covalently) showed a reduction in fluorescence, indicating a decrease in mitochondrial mass. The results are provided in FIG. 3C. This reduction was similar to the one obtained with Urolithin A, a molecule recently reported to induce mitochondrial clearance (Ryu, D., et al., Urolithin A induces mitophagy and prolongs lifespan in C. elegans and increases muscle function in rodents. Nat Med, 2016. 22 (8): p. 879-88).

To corroborate the increase in mitochondrial clearance, we determined mitophagy by testing the compounds in INS1 cells expressing mCherry-GFP, a mitochondrial probe that emits green and red fluorescence under physiologic pH but red fluorescence only under acidic pH (such as that occurring in autolysosomes) and is therefore used for quantifying mitophagy. Unlike MitoTimer, which captures the accumulating effect of mitochondrial turnover, mCherry-GFP captures mitophagy at the time that it occurs. Since the timing of the induction of mitophagy by the hits was not known, we did a time-lapse. The cells were treated with the hits for 2, 8 and 18 hours. While after 2 hours and 18 hours, the molecules tended to increase the number of mitochondria inside autophagosomes (data not shown), only after 8 hours was this increase significant. As shown in FIG. 3D, SPB08007 (compound of structural Formula 6) and MWP00839 increased the number of red fluorescent dots found in acidic vesicles, indicating an increase in mitophagy. This increase was accompanied by an increase in autophagy, as demonstrated in FIG. 3F.

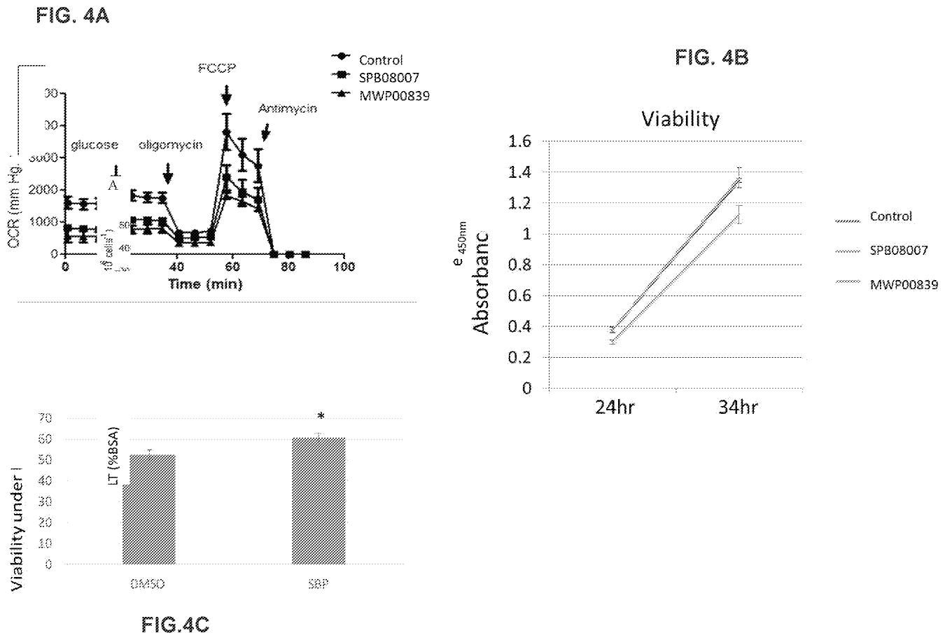

The effect of the hits on respiration was determined using a Seahorse respirometer. As shown in FIG. 4A, 18 hours exposure to both SPB08007 (compound of structural Formula 6) and MWP00839 decreased basal oxygen consumption rate (OCR) and maximal OCR (under FCCP induction). OCR under oligomycin was decreased as well, suggesting a reduction in proton leak. As can be seen from FIG. 4B, SPB08007 did not affect viability after 24 hours exposure while MWP00839 showed a toxic effect.

To address the potential beneficiary effect of mitochondrial clearance by SPB08007 (compound of structural Formula 6), we employed lipotoxicity, a model that we have previously reported to impair autophagy and mitochondrial clearance by alkalization of the lysosomes (Las, G., et al., Fatty acids suppress autophagic turnover in beta-cells. J Biol Chem, 2011.286 (49): p. 42534-44). Co-treatment of INS1 cells with palmitate for 18 hours did not show any protection as compared to INS1 cells treated with palmitate alone, indicating that the effect of SPB08007 occurs upstream to lysosome alkalization. Nevertheless, when the cells were treated with palmitate for 24 hours and were then allowed to recover for 18 hours in the presence or absence of SPB08007, a slight but significant increase in recovery was observed, as can be seen from FIG. 4C.

### Example 5. Further Description of MitoTimer and Screening Methods

The following abbreviations are used herein:

ψₘ—mitochondrial membrane potential

### CI—Mitochondrial Complex I

### CII—Mitochondrial Complex II

### CIII—Mitochondrial Complex III

DMSO—dimethyl sulfoxide
FCCP—Carbonyl cyanide-4-phenylhydrazone

### HCS—High Content Screening

HTS—high-throughput screen

### KRB—Krebs-Ringer Bicarbonate

MTG—Mitotracker green
mtOCR—mitochondrial Oxygen Consumption Rate
mtQC—Mitochondrial Quality Control
mTOR—mechanistic target of rapamycin
NDUFB8—NADH dehydrogenase [ubiquinone] 1 beta subcomplex subunit 8

### OCR—Oxygen Consumption Rate

OXPHOS—Oxidative phosphorylation
PINK1—PTEN induced putative kinase 1
ROS—reactive oxygen species
SDHB—succinate dehydrogenase complex iron sulfur subunit B
TMRE—Tetramethylrhodamine, ethyl ester
UQCRC2—Cytochrome b-c1 complex subunit 2
Z′—Z-prime factor

MitoTimer expressing INS1 cells generation, Cell Culture and treatments. MitoTimer-reporter was cloned into the pHAGE2 lenti-vector under the full-length EF1-alpha promoter to obtain constitutive expression. For the HCS, INS1 cells were infected with lentivirus carrying mitochondrial complex-I-targeted-MitoTimer plasmid. Highly expressing cells were selected by high speed fluorescence activated cell sorter (SY3200 cell sorter, Synergy, iCyt) and kept by the addition of puromycin (1 mg . L-1) in the cell media. Cells were thawed before each experiment, and MitoTimer expression checked through fluorescence.

INS1 cells were cultured with 100 IU/mL penicillin/streptomycin in RPMI media (12 mM glucose, 10% fetal bovine serum, 1 mM pyruvic acid, 10 mM Hepes, 2 mM glutamine and 0.1% beta-mercaptoethanol) at 37° C. and 5% CO2.

High-Content Screening. To test the effects of different known modulators of autophagy and mitophagy on Mitotimer red/green ratio, and therefore determine the best control to use in the HCS accordingly to the estimated z-factor (Zhang, J. H., Chung, T. D., Oldenburg, K. R. (1999). A simple statistical parameter for Use in evaluation and validation of high throughput screening assays. J. Biomol. Screen. 4, 67-73), INS1 cells expressing Mitotimer were treated for 18 h with: urolithin A 50 mM, Bafilomycin A 200 nM, Chloroquine 20 uM, and Rapamycin 100 nM and imaged in two wavelengths (FITC—excitation 482/35 nM, emission 520/35 nm and TRITC—excitation 543/22 nm, emission 593/40 nm) at Operetta Perkin Elmer microscope with 40× lens. HCS assay was developed at the Drug Discovery Unit, INCPM, Weizmann Institute of Science. The screening of 15,000 compounds (LOPAC—pharmacologically active compounds, Maybridge—diverse library, and Analyticon) was performed in INS1 cells stably expressing MitoTimer under EF-1-alpha promoter. Briefly, cells were treated with 10 uM of each compounds for 18 h and imaged in two wavelengths (FITC—excitation 482/35 nM, emission 520/35 nm and TRITC—excitation 543/22 nm, emission 593/40 nm) at ImageXpress Micro High-Content Imaging System/Molecular Devices. Rapamycin 100 nM was used as a control. In each cell plate, the neutral (DMSO) and positive control (Rapamycin) were repeated in 16 replicates while the treatments were performed in duplicates. Each plate was replicated and only hits found in the 2 independent plates were considered. Two fields of each well were analyzed, what normally allowed the analysis of 20-40 cells per condition. MitoTimer red/green ratio for every cell was done in MetaXpress. To establish the hits which affect mitochondrial turnover due to clearance, four criteria were followed based on the calculated z-score (z-score≥0.5) relative to the control (DMSO treated cells): 1) reduced MitoTimer red/green ratio; 2) MitoTimer red/green ratio reduction due to effect on the red readout; 3) green readout not increased more than 0.2 p-value from vehicle d) treatments which resulted in massive cell death excluded. The compounds which affected MitoTimer red/green ratio in a dose response, ranging from 10 to 50 μM, were classified as a hit.

The “screenability” or robustness of a high-throughput screen can be expressed as a z′ factor, which is calculated as follow:

Estimated z-factor (z′) was calculated by:

$${Z}^{‵} = \frac{1 - 3 {c}_{} + {s}_{} )}{\vert {μ}_{c} - {μ}_{s} \vert}$$

where σ and μ are standard deviation and mean, respectively, of control (c) and sample (s).

mCherry-GFP-Fis1₁₀₁₋₁₅₂ determination of mitophagy. INS1 cells were seeded into 4 compartment CELLVIEW™ glass bottom cell culture dishes at a density of 2*104 cells/compartment. After 48 hours cells were transduced with an adenoviral construct encoding the fluorescent mitophagy reporter mCherry-GFP-Fis1₁₀₁₋₁₅₂ for 24 hours. A media change containing the indicated treatment concentration or 0.1% DMSO as vehicle control was performed 8 hours before the imaging session. Imaging was performed using a 63× Plan Neofluar objective and the Airyscan module of a Zeiss LSM880 confocal microscope. 25 visual fields containing 59-85 cells in total were imaged per condition. The experiment was repeated three times independently. Image analysis was performed with FIJI ImageJ 1.51p and CellProfiler 2.2.0 rev ac0529e. Briefly, individual cells were cropped and background was subtracted using a median filter assisted processing method (Dunn, K. W., Kamocka, M. M. McDonald, J. H. (2011) A practical guide to evaluating colocalization in biological microscopy. Am. J. Physiol. Cell Physiol. 300, C723-C742). Ratios of red/green fluorescence channels were computed and mitophagy positive structures were recognized based on a lower ratio cut-off of 2.5 of red over green. Mitophagy events per cell were determined by the following formula.

Mitophagy events per cell=(Total area of mitophagy positive structures per cell)/(Average area of one mitophagy structure)

Data were normalized to DMSO controls to account for day to day variation. Statistical analysis was performed using GraphPad Prism 7.02. Significant treatment differences were determined using One-way ANOVA and Tukey post-hoc analysis. Values of p<0.05 (*) were considered significant.

Mitotracker Green Fluorescence, Anion Radical Superoxide and Mitochondrial Membrane Potential. INS1 cells were seeded into wells of a 96-wells glass bottom cell culture plate (CELLSTAR) at a density of 2*104 cells/well. To assess mitochondrial clearance, a pulse-chase experiment was performed. Briefly, cells were stained with MTG (Molecular Probes) 500 nM for 30 min, washed, and treated with (25 uM) or DMSO 0.1%. After 18 h, cells were imaged using a 40× lens of a Perkin Elmer Operetta high-content imaging system microscope. Results are expressed as the percentage of MTG fluorescence relative to MTG fluorescence of the “time 0” control. To assess mitochondrial mass and anion radical superoxide, INS1 cells were treated with SPB08007, MWP00839 (25 UM) or DMSO 0.1% and after 18 h stained with MTG 500 nM or Mitosox for 30 min or 10 min, respectively, washed and imaged in the same conditions. Integrated fluorescence of each field was analyzed in Image J after background subtraction and threshold (IsoData) application. Twenty-four fields of each condition were analyzed in each experiment.

To assess mitochondrial membrane potential, INS1 cells were plated in each compartment of a quadrant imaging dish (Greiner Bio-One International) at a density of 1.2*105 cells/well. After 48 h, cells were incubated RPMI-1640 containing 25 μM of compound A or vehicle (0.1% DMSO). After 18h, the cells were stained in RPMI-1640 media containing 200 nM MitoTracker Green (MTG) and 15 nM of Tetramethylrhodamine, ethyl ester (TMRE); cells were then washed three times and finally kept in RPMI-1640 containing TMRE but lacking MTG. Live-Imaging was performed using a 63× Plan Neofluar objective and the Airyscan module of a Zeiss LSM880 confocal microscope. The mitochondrial membrane potential was analyzed by TMRE fluorescence corrected by MTG, using the Fiji/ImageJ software. One-way ANOVA and Tukey's multiple comparison test were used for statistical analysis; P-values <0.05 (*) were considered significantly different.

Cellular oxygen consumption. An hour before oxygen consumption measurements, cell media was replaced by assay media (2 mM glucose, 0.8 mM Mg 2+, 1.8 mM Ca 2+, 143 mM NaCl, 5.4 mM KCl, 0.91 mM, NaH 2 PO 4 , and 15 mg/mL Phenol red) for 120 min at 37° C. (no CO2) before loading into the Seahorse Bioscience XF24 extracellular analyzer. The ports of the cartridge containing the oxygen probes were loaded with the compounds to be injected during the assay (50 μL/port) and the cartridge was calibrated.

Basal respiration was recorded for 30 min, at approximately 5 min intervals until system stabilization. Glucose was injected at a final concentration of 12 mM and glucose stimulated respiration was recorded for approximately 15 min. FCCP (Carbonyl cyanide-4-phenylhydrazone) was used at final concentration of 4 μM and injected with sodium pyruvate (Sigma) at a final concentration of 5 mM. Oligomycin and antimycin were used at final concentrations of 2 and 4 μM, respectively. All respiratory modulators were used at ideal concentrations titrated during preliminary experiments (not shown) and oxygen consumption rates were recorded for up to 15 min due the toxicity of these compounds. OCR typical chart is displayed as OCR percentage of basal respiration. All the OCR values were subtracted from the lowest antimycin OCR.

Complex I Activity assay. Mitochondrial complex I enzyme activity was measured using Abcam's complex I enzyme activity microplate assay Kit (ab109721) by following the oxidation of reduced nicotinamide adenine dinucleotide (NADH) to oxidized NAD+ and the simultaneous reduction of a dye which leads to increased absorbance at OD=450 nm.

Cell Viability. Viability was measured using an XTT kit (Biological Industries), as recommended by the manufacturer. 30,000 cells were seeded in a flat 96-well plate. To each well 100 μl of growth media was added. The cells were incubated in a CO2 incubator at 37° C. In most cases, cells can be used to assay proliferation after 24-96 hours. Each test included a blank containing complete medium without cells as a background control. To prepare a reaction solution sufficient for one plate (96 wells), 0.1 ml activation solution was added to 5 ml XTT reagent. 50 μl of the reaction solution was added to each well and the plate was incubated in an incubator for 2-24 hours. Following the incubation, the plate was shacked gently to evenly distribute the dye in the wells and the absorbance of the samples was measured against a background control with a spectrophotometer (ELISA reader) at a wavelength of 480 nm. To determine non-specific readings we used a wavelength of 650 nm which we subtracted from the 480 nm measurement. The average absorbance of the blank control wells was finally subtracted from that of the other wells.

For all the experiment concerning cell viability, a kit of XTT reagent was used (Biological Industries).

Cell treatments and mitochondrial extraction. INS1 cells were treated with SBP08007 or MWP00839 25 uM for 24 h. Mitochondrial and cytosolic fractions from INS1 cells were performed by using the Mitochondria Isolation Kit for Cultured Cells (Pierce). INS1 cells were lysed in a hypotonic buffer (10 mM NaCl, 1.5 mM MgCl2, and 10 mM Tris.HCl, pH 7.5), and mitochondria were extracted in a Dounce homogenizer in mitochondrial buffer (1 mM EDTA, 210 mM mannitol, 70 mM sucrose, and 5 mM Tris.HCl, 7.5), followed by centrifugation at 1,300×g for 10 min at 4° C. The supernatant was further centrifuged at 17,000×g for 15 min at 4° C. to pellet the mitochondria. The crude mitochondrial fraction was resuspended for washing and centrifuged at 17,000×g for 15 min at 4° C. The pellets were collected as the mitochondrial fraction.

Western blot. Cell lysates were diluted in Laemmli sample buffer (100 mM Tris-HCl, 2% SDS, 10% glycerol, 0.1% bromophenol blue) containing 5% beta-mercaptoethanol. After heating at 95° C. for 5 min, proteins were separated by SDS-PAGE previously added of Stain Free reagent (BioRad), and transferred onto PVDF membranes. Membranes were blocked with 5% non-fat milk and detection of individual proteins was carried out by blotting with specific primary antibodies against ubiquitin (Calbiochem 1:1,000). Chemiluminescence detection using a secondary peroxidase-linked anti-rabbit (Calbiochem; 1:10,000) and a detection system from Pierce KLP (Rockford, Ill., USA) was performed. Stain free total protein labeling was imaged in Image Lab 6.0 Software (BioRad), which was used for quantification of the mitochondria and normalization to total protein staining.

Islet isolation. Islets were isolated from DBA/2 mice. Briefly, pancreata of anesthetized mice were infused with collagenase (1 mg/ml, type XI, Sigma-Aldrich, Rehovot, Israel), excised, and incubated for 30 min at 37° C. The digested tissue was vortexed, filtered and washed in HBSS (Biological Industries) containing 0.5% BSA (Sigma). The pellet was resuspended in RPMI medium 1640 supplemented with 10% FCS, 50 units/ml penicillin, and 50 μg/ml streptomycin (all from Biological Industries). Islets were collected on a 100-μm cell strainer (BD, Falcon) and hand-picked using stereoscope (Zeiss, Oberkochen, Germany)

Glucose stimulation insulin secretion assay. After 24-hour pretreatment with SPB08007, 25 uM, islets were washed twice with PBS (Biological Industries, Bet-Haemek, Israel). For basal insulin secretion, the islets were incubated with Krebs-Ringer Bicarbonate (KRB) buffer supplemented with 0.5% BSA and 2 mM glucose for 30 min at 37 deg. C and 5% CO2. For stimulated insulin secretion, the buffer was replaced with KRB buffer supplemented with 0.5% BSA and 16 mM glucose and the islets were incubated for additional 60 min. Supernatant insulin content was measured by ELISA (Merck Millipore, Burlington, Mass.).

### Example 6. MitoTimer Optimization to Follow Mitochondrial Turnover in a High-Content Screen

The aging of the mitochondrial network can be followed through fluorescence switch of MitoTimer from green to red over time. Newly synthesized protein is incorporated into newly formed mitochondria. Where MitoTimer expression is inducible, for example under a doxycycline promoter, a pulse of doxycycline generates an initially green mitochondrial network which, after 16 h, becomes a mix of green and red fluorescence (observable as yellow and orange) and is seen as completely red after 48 h.

We developed a system in which MitoTimer is stably expressed under the EF-1-alpha promoter. In this system MitoTimer is continuously expressed; basal rates of synthesis and degradation of mitochondria, and therefore MitoTimer, are steady, resulting in a constant red/green ratio. The ratiometric analysis corrects for differences in expression levels and gives a more robust index than the comparison of individual integrated fluorescence intensities.

Manipulating mitochondrial turnover alters MitoTimer red/green ratio, in a manner inversely proportional to mitochondrial biogenesis and mitophagy. Therefore, green and red fluorescence intensities must be analyzed individually to conclude which events are impacting the ratio. Changes to MitoTimer green levels reflect protein incorporation into the mitochondria, mainly due to mitochondrial biogenesis or import, while changes in MitoTimer red fluorescence indicate changes in the degradation rate of the protein, if integrated green fluorescence intensity is not altered between experimental groups. Where green and red MitoTimer fluorescence intensities are affected, the ratio may not reflect changes on mitochondrial turnover.

The length of the experiment was adjusted so that conditions affecting green MitoTimer levels did not result in changes in red MitoTimer levels. Once every green protein becomes red after 18-20 h, all cell treatments were performed under 18 h to isolate the effects on MitoTimer red from MitoTimer green readouts. This is particularly important in mitophagy studies since, in most cases, mitochondrial biogenesis increases in parallel to mitophagy.

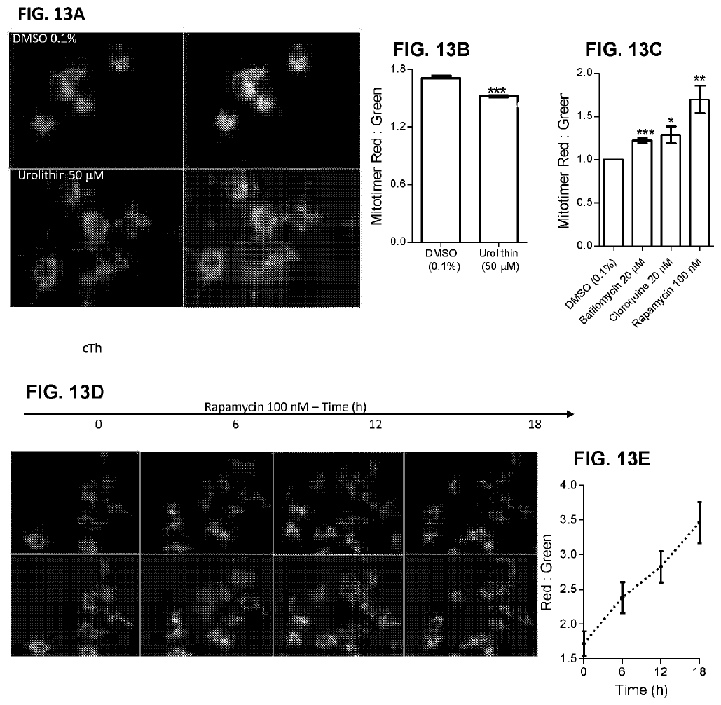

We tested three classes of compounds as controls to establish MitoTimer red/green ratio as an index to follow mitochondrial turnover in a High-Content Screen (HCS) aiming to find the best control for the screening: (1) Urolithin A, a natural compound discovered as a stimulator of mitophagy; (2) inhibitors of macro-autophagy, bafilomycin A and chloroquine, and (3) rapamycin, a classical mTOR (mechanistic target of rapamycin) inhibitor which stimulates macroautophagy in most cell types. Urolithin A significantly reduced MitoTimer red/green ratio in INS1 cells (FIGS. 13A and B), due to reduction in red fluorescence, indicating enhanced mitochondrial clearance. Preventing lysosome acidification and fusion with autophagosome as a result of bafilomycin A1 treatment enhanced the population of red mitochondria, and therefore, increased red/green MitoTimer ratio; the same was observed when neutralizing lysosome acidity with chloroquine (FIG. 13C). We previously demonstrated that inhibiting these last steps of autophagy affects MitoTimer red/green ratio readout in MEF cells. Rapamycin increased MitoTimer red/green ratio by 50% (FIG. 13C), due to an accumulation of red fluorescence over time, resulting in a time dependent increase in red/green ratio (FIGS. 13 D and E).

Although macroautophagy is required for the complete elimination of mitochondria and its inhibition certainly impairs basal mitophagy in some cell types, there is no evidence the stimulation of macroautophagy in the absence of mitochondrial stress can increase mitophagy, a selective event which require loss of ψm and segregation of the damage units from the network. In fact, it has been demonstrated that autophagy activation by nutrient starvation or rapamycin impairs mitophagy due to mitochondrial elongation and inability to segregate.

Rapamycin effect on MitoTimer red/green ratio was the strongest among all controls tested, and provided an average prime Z factor (Z′) relative to vehicle, DMSO (dimethyl sulfoxide) of 0.4 indicating a robust response, therefore, Rapamycin was used as a positive control in every cell plate. Briefly, z′ expresses how effectively the assay separates the positive and negative control values, based on means and standard deviations of both controls (full formula on methodology section), and it indicates the “screenability” of a high-throughput screen (HTS). The closer to 1 the z′ is, the more robust the assay is.

Z-score was calculated for the experimental conditions; in this case, it determines how far a testing molecule is from the control, indicating the quality of the assay and providing a cutoff to establish hits in a HCS/HTS. In the present HCS, we focused on the search for molecules that enhance mitochondrial turnover (lower red/green MitoTimer ratio, with reduced red MitoTimer levels). To determine a small molecule is a hit, red/green MitoTimer ratio and red fluorescence reduction z-score was set to be higher than 0.5 from DMSO; and green fluorescence and cell number variation of less than 0.5 z-score from DMSO control (FIG. 5B).

### Example 7. Identification of 2 Chemically-related Compounds that Stimulate Basal Mitochondrial Turnover Through Mitophagy

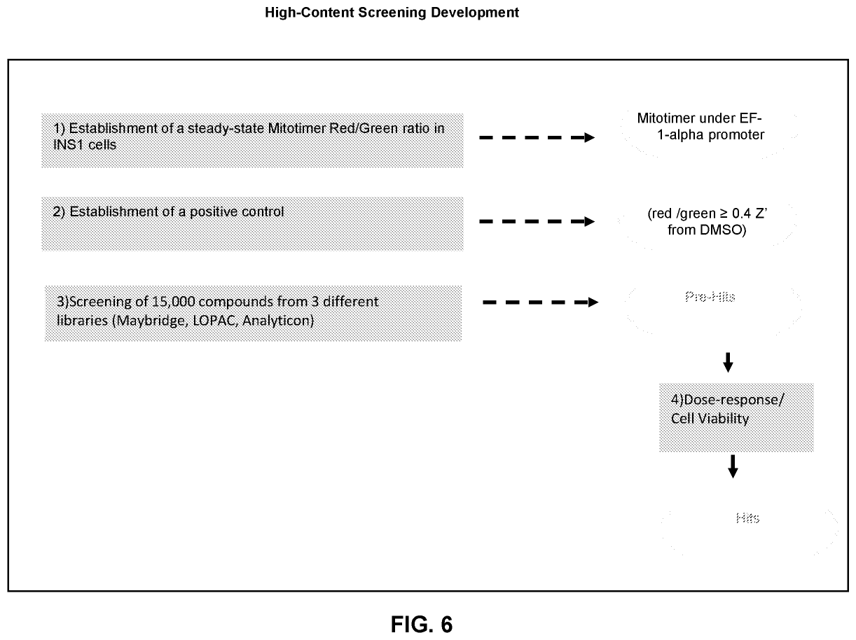

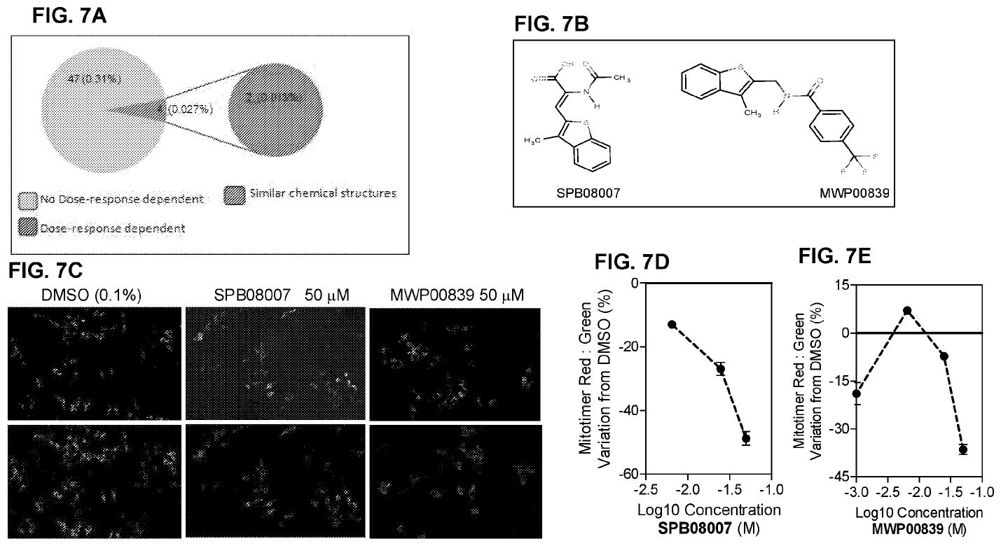

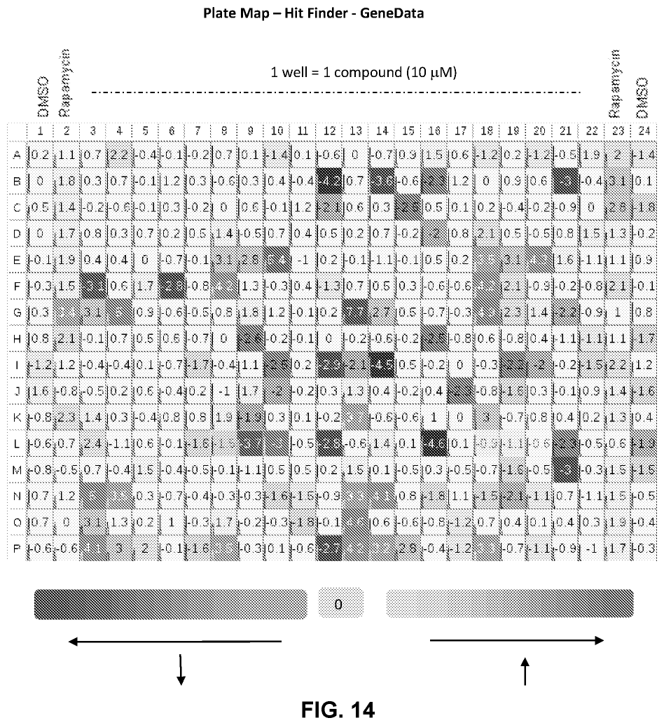

The HCS steps are summarized in FIG. 6. Briefly, after establishing the best expression system which allowed a steady state red/green MitoTimer ratio in the controls for the screening, INS1 cells were plated in 384-well plates, and in each well, 10 uM of a given compound were added for 16-18 h and live-cell imaging was performed using FITC (excitation 482/35 nm, emission 520/35 nm) and TRITC (excitation 543/22 nm, emission 593/40 nm) channels. The small molecules were chosen from 3 libraries: LOPAC—pharmacologically active compounds, Maybridge—diverse library, and Analyticon. Individual cells were identified using the red channel, and red and green MitoTimer fluorescence were analyzed and ratio determined per cell. In the Hit finder software (GeneData), shown in FIG. 14, the small molecules which impacted mitochondrial turnover based on the chosen parameters (FIG. 5B) were identified. From 15,000 compounds, 47 reduced the red/green MitoTimer ratio, with an effect on red MitoTimer levels (FIG. 4A). The molecules that acted in a dose-response manner (4, 0.027%) were classified as the final hits. Amongst those, two benzothiophene derivatives from the Maybridge library, SPB08007 and MWP00839, are structurally similar (FIG. 7B) and were further investigated.

SPB08007 and MWP00839 at 10 uM reduced red/green MitoTimer ratio (FIGS. 7 C and D), due to lowering the levels of red MitoTimer. These results indicate a higher mitochondrial turnover rate due to mitochondrial clearance. The maximum reduction on the ratio index was 40 and 60%, respectively, at 50 uM dose (FIGS. 7 B, C and D), which also reflected an increase in MitoTimer green readout. Mitophagy and mitochondrial biogenesis have been reported to be activated in parallel and at the highest concentrations, SPB08007 and MWP00839 are likely stimulating the formation of new mitochondria as well.

The impact of the hits on mitochondrial turnover was further confirmed by a mitophagy-specific assay, a protein with a tandem mCherry-GFP tag attached to the localization signal of the protein FIS at the OMM. Mitochondria of cells expressing this protein fluoresce red and green. When mitochondria enter the lysosomes, the GFP fluorescence is quenched, the remaining red fluorescence is seen as red puncta, representing the undergoing mitophagy events.

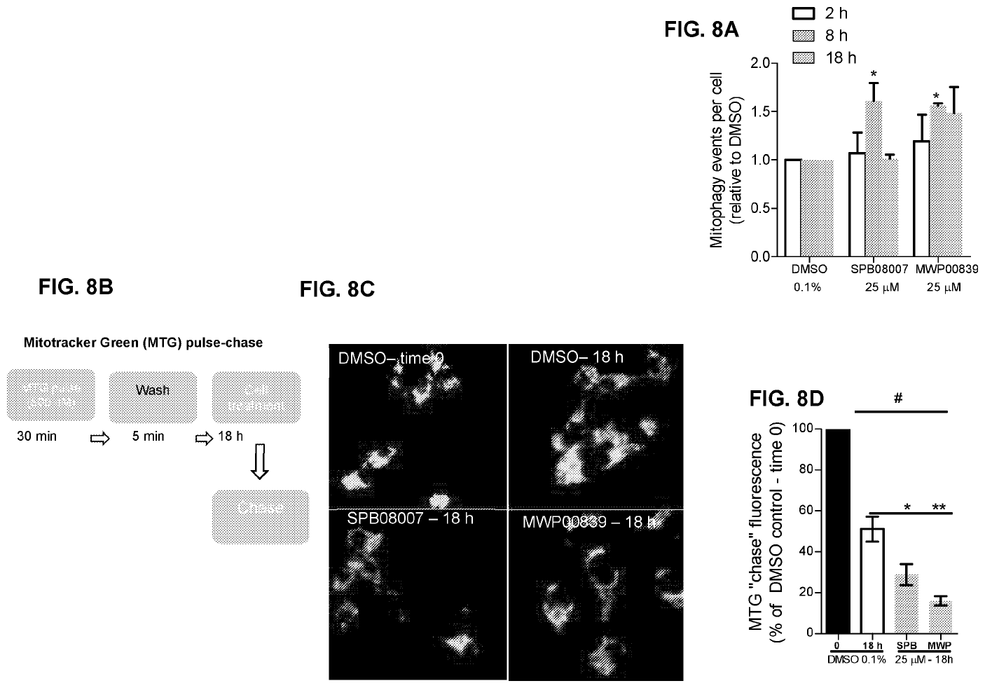

INS1 cells treatment with SPB08007 and MWP00839 significantly increased the number of acidic puncta (FIGS. 8A and B). Interestingly, the formation of acidic puncta reached a peak at 8 h (FIG. 8A). In cells treated with MWP00839, the number of mitophagy events was sustained at 18 h, whereas in cells treated with SPB08007 mitophagy returned to control levels at 18 h, suggesting basal mitophagy can be stimulated, but is tightly regulated (FIG. 8 A). Basal mitophagy is currently being explored and, contrary to stress-induced mitophagy, was unveiled to be independent of PINK1 in most mammalian and Drosophila melanogaster tissues.

A second fluorescence technique, Mitotracker Green (MTG) “pulse-and-chase” (FIG. 8 C-E), was utilized to confirm faster mitochondrial clearance upon treatment with SPB08007 and MWP00839. After a pulse with the mitochondrial labeling probe MTG, INS1 cells were incubated with SPB08007 and MWP00839 and MTG fluorescence was chased after 18 h. The “pulse-chase” experiment allowed the quantification of the pool of mitochondria stained during the pulse and remaining after the 18 h treatment period. Control cells at “time zero” were stained with MTG before imaging and represented the 100% baseline. As expected, all groups had lower MTG content after 18 h of the pulse compared to control “time zero” and both SPB08007 and MWP00839 treatments resulted in lower MTG intensity compared to DMSO (FIGS. 8 D and E). These findings further confirm higher mitochondrial clearance upon treatment with SPB08007 and MWP00839. No differences in cell replication, which could account for MTG dilution, were observed between the groups (data not shown). At the dose used for most of the assays, the compounds conserved the cellular viability, and SPB08007 effects were comparable to DMSO control (FIG. 4B).

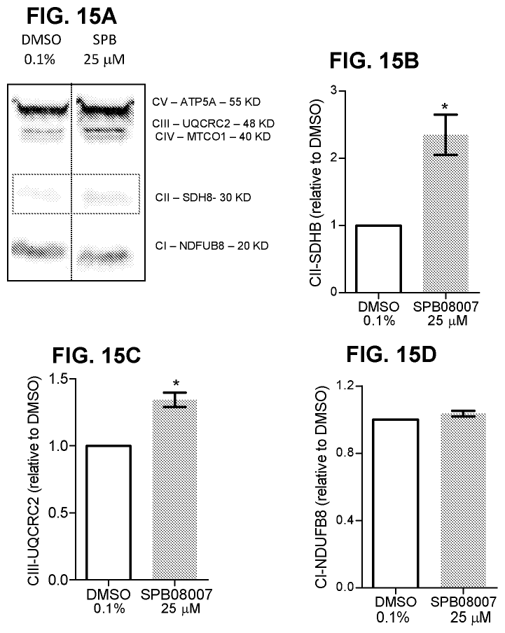

The degradation rate of mitochondrial proteins varies between them and differs from mitochondria as a whole, therefore, following specific mitochondrial proteins is a limited approach to infer changes in mitochondrial mass or turnover. Nonetheless, C-II protein SDHB (succinate dehydrogenase complex iron sulfur subunit B, Complex II) and C-III protein UQCRC2 (Cytochrome b-c1 complex subunit 2) were increased by 2 and 1.5 fold, respectively, (FIG. 15), similarly to what was previously observed following stimulation of mitophagy in C2C12 myotubes with Urolithin A. CI—NDFUB8 levels (FIGS. 15A and D) were unaffected by SPB08007.

### Example 8. SPB08007 Improves Mitochondrial Efficiency and Complex I Activity

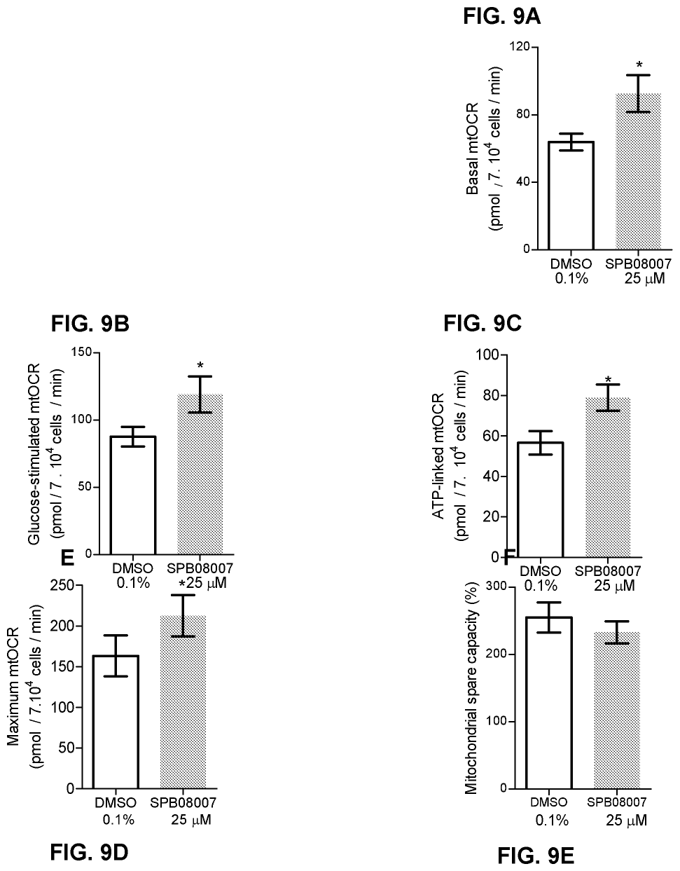

SPB08007 increased basal (FIG. 9A, B), glucose-stimulated (FIG. 9A, C) and maximum (FIG. 9A, E) mitochondrial oxygen consumption rates (mtOCRs), while conserving mitochondrial coupling (oligomycin-sensitive respiration), resulting in higher ATP-linked oxygen consumption (FIG. 9A, D). In the absence of an effect on mitochondrial coupling or increased mitochondrial mass, higher basal respiration is normally a response to a higher energy demand, which can be triggered by mitophagy/biogenesis events and by other unknown cellular processes activated by this compound. The spare respiratory capacity (increase of mtOCR caused by the uncoupler FCCP) relative to basal respiration was unchanged by compound A, despite higher absolute values of mtOCR (FIG. 9A, E), which indicates that mitochondria are using more of their respiratory reserve compared to the control, probably due to a higher energetic demand.

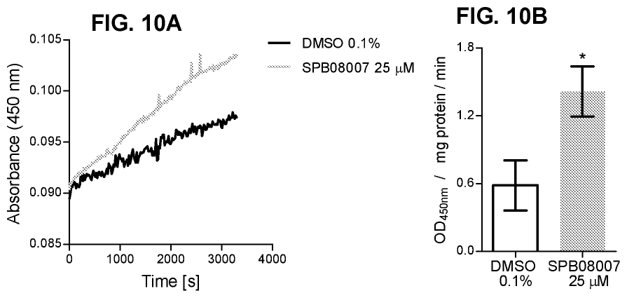

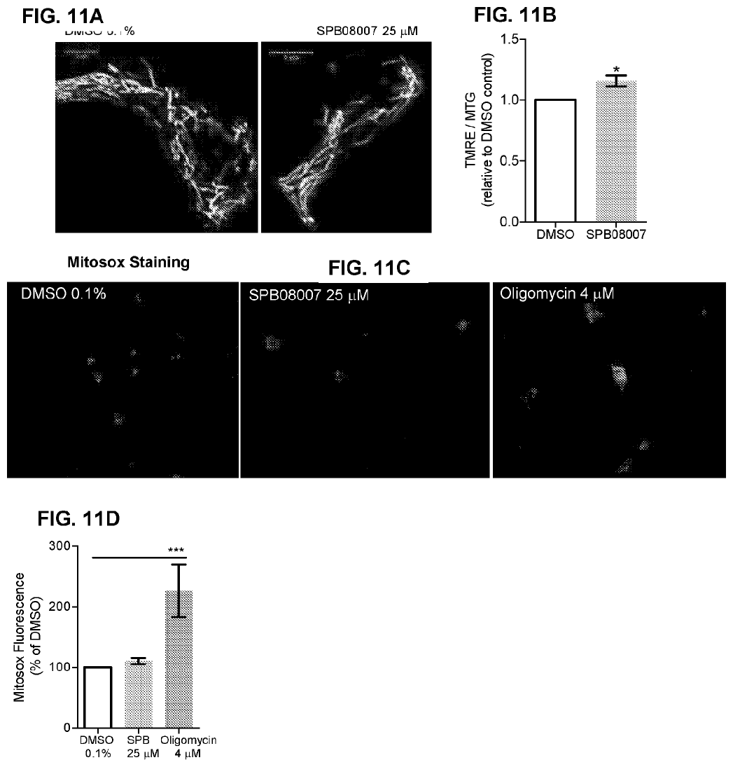

The higher mitochondrial efficiency was associated with a two-and-a-half-fold increase in mitochondrial Complex I activity (FIG. 10A, B), which contributes to a faster flow of electrons (FIG. 7). The ψm was not reduced by SPB08007 (FIG. 11A, B), strongly indicating this drug mechanism of action is not through loss of ψm. Indeed, anion radical superoxide formation, measured by Mitosox, was unaffected (FIG. 11 C, D). Thus, unlike other known mitophagy activators, SPB08007 is not causing mitochondrial stress or uncoupling.

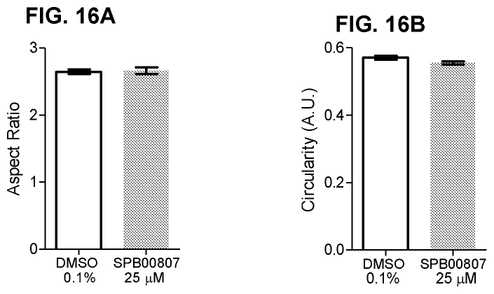

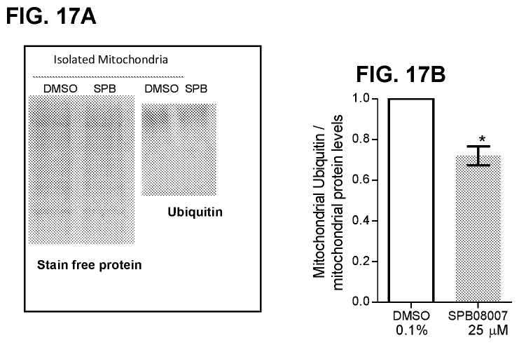

In fact, the ψm was slightly, but significantly, raised by this drug, without parallel changes in mitochondrial morphology (FIG. 16). The ongoing basal mitophagy activation is likely eliminating the mitochondrial subpopulation with lower potential, as expected, increasing the pool of coupled mitochondria, with a higher average TMRE/MTG ratio (FIG. 11A, B). The improved CI activity, without changes in CI protein levels, may also reflect the resulting concentration of “young mitochondria” (less damaged), as indicated by lower MitoTimer red/green ratio (FIG. 7). Interestingly, mitochondrial proteins isolated from INS1 cells previously treated with SPB08007, were found to be less ubiquitinated (FIG. 17), suggesting SPB08007 is not causing mitochondrial ubiquitination, but it is rather improving the elimination of organelles with higher ubiquitin content.

### Example 9. SPB08007 Decreases Basal Insulin Secretion

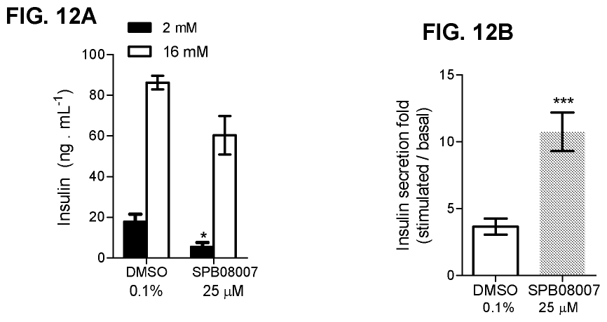

Elevated basal insulin secretion under fasting conditions together with insufficient stimulated insulin release is an important hallmark of type 2 diabetes. In vitro, high basal insulin secretion has been linked to mitochondrial dysfunction, for example in response to glucolipotoxicity and PINK1 deficiency. SPB08007 significantly reduced insulin secretion at 2 mM glucose (basal), while maintaining the levels of glucose-stimulated secretion (16 mM of glucose) (FIG. 12A), resulting in a higher secretion fold in response to glucose (FIG. 12 B), which reflects a better metabolic coupling. We previously found similar results in mouse islets treated with serum of calorie-restricted animals, an in vitro model associated with extended cellular lifespan and improved mitochondrial function.

Overall, by establishing MitoTimer as a feasible tool to follow mitochondrial turnover in a HTS/HCS, and focusing on mitochondrial clearance, we found 2 chemically-related small molecules that enhance basal mitophagy without causing mitochondrial stress. The existence of a “spare basal mitophagy” which can be activated in a physiological context presents an exciting, new model for studying mitophagy and suggests that novel regulators of this pathway could be viable targets for future drug development.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising 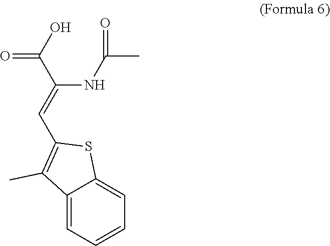 [SMILES: CC(=O)N/C(=C\c1sc2ccccc2c1C)C(=O)O]
or any of its stereoisomers, or any mixture of its stereoisomers, or a pharmaceutically acceptable salt thereof, and a pharmaceutically-acceptable excipient.

2. A nanoparticle comprising the compound of Formula 6 or the pharmaceutical composition of claim 1.

3. A method for treating a subject having a disease or disorder associated with impaired mitochondrial clearance or having a disease or disorder associated with impaired mitochondrial turnover comprising administering to the subject an effective amount of the compound of Formula 6 or the pharmaceutical composition of claim 1.

4. A method for treating a subject having a disease or disorder selected from the group consisting of age-related diseases, age-related disorders, neurodegenerative diseases, neurodegenerative disorders, Alzheimer's disease, Parkinson's disease, Huntington's disease, and Diabetes mellitus type 2, comprising administering to the subject an effective amount of the compound of Formula 6 or the pharmaceutical composition of claim 1.

5. Use of a compound having Formula 6 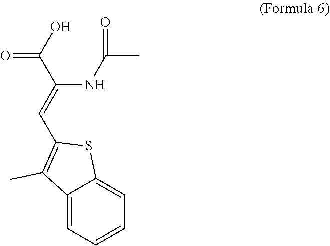 [SMILES: CC(=O)N/C(=C\c1sc2ccccc2c1C)C(=O)O]
in the preparation of a medicament for treating a disease or disorder associated with impaired mitochondrial clearance or for treating a disease or disorder associated with impaired mitochondrial turnover.

6. The use according to claim 5, wherein the medicament for treating a disease or disorder is a disease or disorder selected from the list consisting of age-related diseases, age-related disorders, neurodegenerative diseases, neurodegenerative disorders, Alzheimer's disease, Parkinson's disease, Huntington's disease, and Diabetes mellitus type 2.

7. A compound of the general Formula (I): 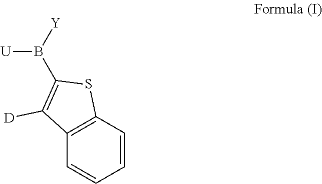
wherein:
D is a functional group selected from a C₁₋ₙ-alkyl, wherein n is an integer from 2 to 5, inclusive, or a substituted alkyl C₁₋ₙ-alkyl -R¹, wherein R¹ is a functional group selected from hydrogen, halo, haloalkyl, cyano, nitro, hydroxyl, alkyl, alkenyl, aryl, alkoxyl, aryloxyl, aralkoxyl, amino, alkylamino, arylamino, aminocarbonyl, carboxyl, or heteroaryl; preferably D is a C₁₋ₙ-alkyl, wherein n is an integer from 2 to 5, inclusive;
B is a linking group selected from a C₂-alkenyl or a C₂-alkynyl, or a substituted C₂-alkenyl-R², wherein R² is a functional group selected from hydrogen, halo, haloalkyl, cyano, nitro, hydroxyl, alkyl, alkenyl, aryl, amino, alkylamino, arylamino and carboxyl; preferably B is a C₂-alkenyl group or a C₂-alkynyl group;
Y is a functional alkylamino group C₁₋ₙ-alkyl-NR³R⁴, wherein n is an integer from 2 to 5, inclusive, wherein R³ and R⁴ are each independently selected from carbonyl, halo, haloalkyl, cyano, nitro, hydroxyl, alkyl, alkenyl, aryl, alkoxyl, amino, alkylamino, arylamino, aminocarbonyl, and carboxyl; preferably R³ is carboxyl and R⁴ is carbonyl;
U may be absent or present, but if present is a functional group selected from hydrogen, a C₁₋ₙ-alkyl, wherein n is an integer from 2 to 5, inclusive, or a substituted alkyl group C₁₋ₙ-alkyl-R⁵, wherein R⁵ is a functional group selected from hydrogen, halo, haloalkyl, cyano, carboxyl, nitro, hydroxyl, alkyl, alkenyl, and aryl; preferably U is hydrogen or absent;
optionally U is absent, and Y and the terminal carbon atom of the B group are forming a closed heterocyclic ring, wherein at least one of the atoms of the ring is nitrogen, and at least one of the atoms of the ring is oxygen; preferably said heterocyclic ring is substituted with one or more groups independently selected from a C₁₋ₙ-alkyl, wherein n is an integer from 2 to 5, inclusive, a substituted alkyl C₁₋ₙ-alkyl-R⁶, wherein n is an integer from 2 to 5, inclusive, and wherein R⁶ is a functional group selected from halo, oxo, haloalkyl, cyano, carbonyl, carboxyl, nitro, hydroxyl, alkyl, alkenyl, aryl; preferably at least two carbon atoms of said heterocyclic ring are substituted, wherein each of the substituents is independently selected from the list provided for R⁶; preferably one of the carbon atoms of said heterocyclic ring is substituted with a C₁₋ₙ-alkyl, wherein n is an integer from 2 to 5, inclusive, and the other carbon atom of said heterocyclic ring is substituted with an oxo (‘═O’) group; or any of its stereoisomers, or any mixture of its stereoisomers, or a pharmaceutically acceptable salt thereof.

8. A compound of the general Formula (II): 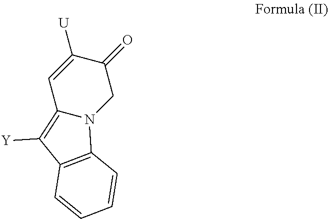
wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁₋ₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

9. A compound of the general Formula (III): 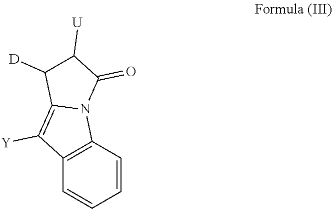
wherein:
Y is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably Y is a C₁₋ₙ-alkyl, wherein n is an integer from 2 to 5, inclusive;
D is a functional group selected from a C₁₋ₙ-alkyl, wherein n is an integer from 2 to 5, inclusive, or a substituted alkyl C₁₋ₙ-alkyl-R¹, wherein R¹ is a functional group selected from hydrogen, halo, haloalkyl, cyano, nitro, hydroxyl, alkyl, alkenyl, aryl, amino, alkylamino, arylamino, aminocarbonyl, carboxyl, and heteroaryl; preferably D is a C₁₋ₙ-alkyl, wherein n is an integer from 2 to 5, inclusive;
U is a functional group selected from carbonyl, carboxyl, halo, haloalkyl, cyano, nitro, hydroxyl, alkyl, alkenyl, aryl, alkoxyl, amino, alkylamino, arylamino, and aminocarbonyl; preferably U is carboxyl;
or any of its stereoisomers, or any mixture of its stereoisomers, or a pharmaceutically acceptable salt thereof.

10. The compound of claim 7 having the Formula 1 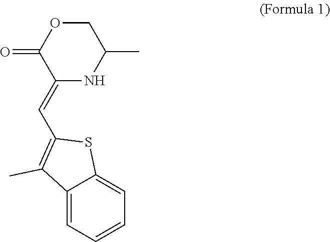 [SMILES: Cc1c(/C=C2\NC(C)COC2=O)sc2ccccc12]
or any of its stereoisomers, or any mixture of its stereoisomers, or a pharmaceutically acceptable salt thereof.

11. The compound of claim 7 having the Formula 2 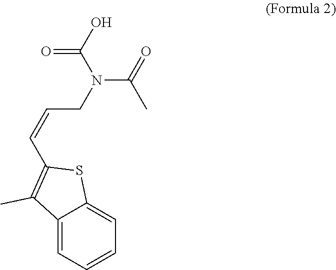 [SMILES: CC(=O)N(C/C=C\c1sc2ccccc2c1C)C(=O)O]
or any of its stereoisomers, or any mixture of its stereoisomers, or a pharmaceutically acceptable salt thereof.

12. The compound of claim 7 having the Formula 3 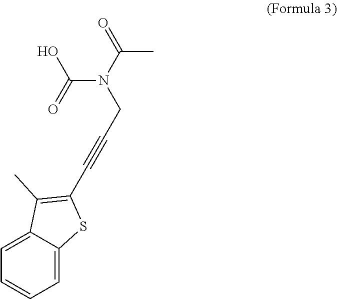 [SMILES: CC(=O)N(CC#Cc1sc2ccccc2c1C)C(=O)O]
or any of its stereoisomers, or any mixture of its stereoisomers, or a pharmaceutically acceptable salt thereof.

13. The compound of claim 8 having the Formula 4 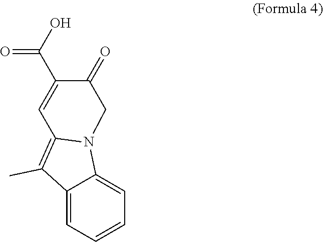 [SMILES: Cc1c2n(c3ccccc13)CC(=O)C(C(=O)O)=C2]
or any of its stereoisomers, or any mixture of its stereoisomers, or a pharmaceutically acceptable salt thereof.

14. The compound of claim 9 having the Formula 5 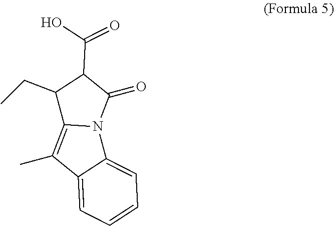 [SMILES: CCC1c2c(C)c3ccccc3n2C(=O)C1C(=O)O]
or any of its stereoisomers, or any mixture of its stereoisomers, or a pharmaceutically acceptable salt thereof.

15. A pharmaceutical composition comprising a compound according to any one of claims 7 to 14 together with one or more pharmaceutically acceptable excipients.

16. Use of a compound according to any one of claims 7 to 14 in the preparation of a medicament.

17. Use of a compound according to any one of claims 7 to 14 for preparing a medicament for treating a disease or disorder associated with impaired mitochondrial clearance or for treating a disease or disorder associated with impaired mitochondrial turnover.

18. Use of a compound according to any one of claims 7 to 14 for preparing a medicament for treating a disease or disorder a disease or disorder selected from the list consisting of age-related diseases, age-related disorders, neurodegenerative diseases, neurodegenerative disorders, Alzheimer's disease, Parkinson's disease, Huntington's disease, and Diabetes mellitus type 2.

19. A method for treating a subject having a disease or disorder associated with impaired mitochondrial clearance or for treating a disease or disorder associated with impaired mitochondrial turnover comprising administering to the subject an effective amount of the compound of any one of claims 7 to 14 or the pharmaceutical composition of claim 14.

20. A method for preventing and/or treating a subject having a disease or disorder selected from the group consisting of age-related diseases, neurodegenerative disorders, neurodegenerative diseases, neurodegenerative disorders, Alzheimer's disease, Parkinson's disease, Huntington's disease, and Diabetes mellitus type 2, comprising administering to the subject in need thereof a therapeutically effective amount of the compound of any one of claims 7-14 or the pharmaceutical composition of claim 15.
